SEQUENCE LISTING (44 sequences)

SEQ ID NO: 1 - protein, 487 aa, synthetic construct
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 22 aa, synthetic construct
GSGATNFSLLKQAGDVEENPGP

SEQ ID NO: 3 - protein, 17 aa, synthetic construct
MLLSVPLLLGLLGLAVA

SEQ ID NO: 4 - protein, 29 aa, synthetic construct
MRISKPHLRSISIQCYLCLLLNSHFLTEA

SEQ ID NO: 5 - protein, 19 aa, synthetic construct
GIHVFILGCFSAGLPKTEA

SEQ ID NO: 6 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 7 - protein, 5 aa, synthetic construct
GGGGS

SEQ ID NO: 8 - protein, 7 aa, synthetic construct
SGGGSGG

SEQ ID NO: 9 - protein, 25 aa, synthetic construct
SGGGSGGGGSGGGSGGGGSGGGSLQ

SEQ ID NO: 10 - protein, 65 aa, synthetic construct
ITCPPPMSVEHADIWVKSYSLYSRERYICNSGFKRKAGTSSLTECVLNKATNVAHWTTPSLKCIR

SEQ ID NO: 11 - protein, 237 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 12 - protein, 4 aa, synthetic construct
KDEL

SEQ ID NO: 13 - protein, 4 aa, synthetic construct
KKYL

SEQ ID NO: 14 - protein, 10 aa, synthetic construct
MGCVCSSNPE

SEQ ID NO: 15 - protein, 7 aa, synthetic construct
MGKAQSK

SEQ ID NO: 16 - protein, 9 aa, synthetic construct
MGCTLSAGS

SEQ ID NO: 17 - protein, 147 aa, synthetic construct
MLLSVPLLLGLLGLAVAGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGKKYL

SEQ ID NO: 18 - protein, 409 aa, synthetic construct
MRISKPHLRSISIQCYLCLLLNSHFLTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHLKDEL

SEQ ID NO: 19 - protein, 402 aa, synthetic construct
MLLSVPLLLGLLGLAVAGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHLKDEL

SEQ ID NO: 20 - protein, 230 aa, synthetic construct
MLLSVPLLLGLLGLAVAGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGSGGGGSGGGSLQITCPPPMSVEHADIWVKSYSLYSRERYICNSGFKRKAGTSSLTECVLNKATNVAHWTTPSLKCIRKDEL

SEQ ID NO: 21 - protein, 415 aa, synthetic construct
MRISKPHLRSISIQCYLCLLLNSHFLTEAMGCVCSSNPENWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 22 - protein, 386 aa, synthetic construct
MGCVCSSNPENWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 23 - protein, 412 aa, synthetic construct
MRISKPHLRSISIQCYLCLLLNSHFLTEAMGKAQSKNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 24 - protein, 414 aa, synthetic construct
MRISKPHLRSISIQCYLCLLLNSHFLTEAMGCTLSAGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 25 - protein, 114 aa, synthetic construct; identical to SEQ ID NO: 6

SEQ ID NO: 26 - protein, 162 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 27 - protein, 119 aa, synthetic construct
AVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSS

SEQ ID NO: 28 - protein, 117 aa, synthetic construct
QVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSS

SEQ ID NO: 29 - protein, 5 aa, synthetic construct
TYFMA

SEQ ID NO: 30 - protein, 17 aa, synthetic construct
GIAWSGGSTAYADSVKG

SEQ ID NO: 31 - protein, 10 aa, synthetic construct
RGIEVEEFGA

SEQ ID NO: 32 - protein, 5 aa, synthetic construct
NYYMG

SEQ ID NO: 33 - protein, 16 aa, synthetic construct
IISSDTTITYKDAVKG

SEQ ID NO: 34 - protein, 9 aa, synthetic construct
WTSDWSVAY

SEQ ID NO: 35 - protein, 5 aa, synthetic construct
GSGGS

SEQ ID NO: 36 - protein, 4 aa, synthetic construct
GGGS

SEQ ID NO: 37 - protein, 4 aa, synthetic construct
GGSG

SEQ ID NO: 38 - protein, 5 aa, synthetic construct
GGSGG

SEQ ID NO: 39 - protein, 5 aa, synthetic construct
GSGSG

SEQ ID NO: 40 - protein, 5 aa, synthetic construct
GSGGG

SEQ ID NO: 41 - protein, 4 aa, synthetic construct
GGSG

SEQ ID NO: 42 - protein, 5 aa, synthetic construct
GSSSG

SEQ ID NO: 43 - protein, 4 aa, synthetic construct
HDEL

SEQ ID NO: 44 - protein, 4 aa, synthetic construct
KDEL

(19) United States
(12) Patent Application Publication
(10) Pub. No.: US 2026/0315594 A1
(43) Pub. Date: Oct. 15, 2026

(54) FUSION PROTEINS AND METHODS OF USE THEREOF

(71) Applicants: LEGEND BIOTECH USA INC., Somerset, NJ (US); Legend Biotech Ireland Limited, Dublin 18 (IE)

(72) Inventors: Tianjian LI, Somerset, NJ (US); Sourindra Maiti, Somerset, NJ (US); Rohit Mathur, Somerset, NJ (US)

(21) Appl. No.: 19/476,596

(22) PCT Filed: May 16, 2024
(86) PCT No.: PCT/US2024/029745

§ 371 Date: Oct. 17, 2025

Related U.S. Application Data
(60) Provisional application No. 63/467,123, filed on May 17, 2023.

(51) Int. Cl. A61K 40/35; A61K 40/15; A61K 40/31; A61K 40/42; A61P 35/00; C07K 14/47; C07K 14/54; C07K 14/705; C07K 14/715; C07K 16/28; C12N 5/0783; C12N 15/86

(52) U.S. Cl. CPC A61K 40/35; A61K 40/15; A61K 40/31; A61K 40/4215; A61P 35/00; C07K 14/4702; C07K 14/5443; C07K 14/7051; C07K 14/7155; C07K 16/2878; C12N 5/0646; C12N 15/86; C07K 2319/02; C12N 2510/00; C12N 2740/10043

(57) ABSTRACT

Provided are fusion proteins comprising an interleukin-15 (IL-15) sequence and an interleukin-15 receptor alpha (IL-15Ra) sequence, with an endoplasmic reticulum (ER) retention sequence and/or a myristoylation sequence.

### CROSS REFERENCE TO RELATED APPLICATION

This application claims benefit of priority of U.S. Patent Application No. U.S. 63/467,123 filed on May 17, 2023, the content of which is incorporated herein by reference in its entirety.

### SEQUENCE STATEMENT

This application contains a Sequence Listing that has been submitted electronically as an XML file named “51624-0073WO1_SL_ST26.XML.” The XML file, created on May 16, 2024, is 43,151 bytes in size. The material in the XML file is hereby incorporated by reference in its entirety.

### TECHNICAL FIELD

This disclosure relates to fusion proteins comprising an interleukin-15 (IL-15) sequence and an interleukin-15 receptor alpha (IL-15Rα) sequence, with an endoplasmic reticulum (ER) retention sequence and/or a myristoylation sequence.

### BACKGROUND

IL-15 is an important cytokine for immune cell function. IL-15 is essential for the survival, proliferation, and functional integrity of NK cells. It can enhance NK cell-mediated immunotherapies. IL-15 is minimally secreted but is effectively delivered by trans-presentation in association with its unique receptor alpha (IL-15 Rα) on the surface of IL-15 producing cells to interact with a receptor complex composed of the IL-2Rβ and common γ chains on target cells.

IL-15 can improve persistence of Natural Killer (NK) cells. However, recombinant IL-15 has a short half-life and thus requires continuous dosing which could lead to undesired side effects. One strategy is to modify the NK cells to express IL-15 to improve persistence. Initial in-vivo results indicated that use of wild type IL-15 in either membrane bound or soluble form in the context of genetically modified NK cells leads to lethality in mice. In addition, it has proven difficult to efficiently culture and expand these cells subjects who have cancer. There is a need to increase the proliferation and anti-tumor efficacy of these immune cells.

### SUMMARY

This disclosure relates to fusion proteins comprising an IL-15 sequence and an IL-15Rα sequence, with an endoplasmic reticulum (ER) retention sequence and/or a myristoylation sequence.

In one aspect, the present disclosure is related to a fusion protein comprising (1) a signal peptide sequence; (2) an interleukin-15 (IL-15) sequence; and (3) an endoplasmic reticulum (ER) retention sequence.

In some embodiments, the signal peptide sequence is calreticulin signal peptide sequence.

In some embodiments, the calreticulin signal peptide sequence comprising an amino acid sequence set forth in SEQ ID NO: 3, or an amino acid sequence having at least 90%, 95%, or 99% identity to the amino acid sequence set forth in SEQ ID NO: 3.

In some embodiments, the ER retention sequence comprises an amino acid sequence set forth in SEQ ID NO: 12 or 13, or an amino acid sequence having at least 90%, 95%, or 99% identity to the amino acid sequence set forth in SEQ ID NO: 12 or 13.

In one aspect, the present disclosure is related to a fusion protein comprising (1) a cell membrane targeting sequence; and (2) an IL-15 sequence.

In some embodiments, the cell membrane targeting sequence is selected from the group consisting of a myristoylation sequence, a palmitoylation sequence, and a prenylation sequence.

In some embodiments, the cell membrane targeting sequence is a myristoylation sequence.

In some embodiments, the myristoylation sequence comprises an amino acid sequence set forth in any one of SEQ ID NOs: 14-16, or an amino acid sequence having at least 90%, 95%, or 99% identity to the amino acid sequence set forth in any one of SEQ ID NOs: 14-16.

In some embodiments, the IL-15 sequence comprises an amino acid sequence set forth in SEQ ID NO: 6, or an amino acid sequence having at least 90%, 95%, or 99% identity to the amino acid sequence set forth in SEQ ID NO: 6.

In some embodiments, the fusion protein further comprises an IL-15Rα sequence.

In some embodiments, the IL-15Rα sequence comprises an amino acid sequence set forth in SEQ ID NO: 11, or an amino acid sequence having at least 90%, 95%, or 99% identity to the amino acid sequence set forth in SEQ ID NO: 11.

In some embodiments, the IL-15Rα sequence comprises or consists of an IL-15Rα Sushi domain, wherein the IL-15Rα Sushi domain comprises or consists of an amino acid sequence set forth in SEQ ID NO: 10, or an amino acid sequence having at least 90%, 95%, or 99% identity to the amino acid sequence set forth in SEQ ID NO: 10.

In some embodiments, the fusion protein comprises, from N-terminus to C-terminus, a signal peptide sequence, a first linker sequence, an IL-15 sequence, a second linker sequence, an IL-15Rα sequence, and an ER retention sequence.

In some embodiments, the fusion protein comprises an amino acid sequence set forth in any one of SEQ ID NOs: 18-20, or an amino acid sequence having at least 90%, 95%, or 99% identity to the amino acid sequence set forth in any one of SEQ ID NOs: 18-20.

In some embodiments, the fusion protein comprises an amino acid sequence set forth in SEQ ID NO: 19, or an amino acid sequence having at least 90%, 95%, or 99% identity to the amino acid sequence set forth in SEQ ID NO: 19.

In some embodiments, the fusion protein comprises, from N-terminus to C-terminus, an optional signal peptide sequence, a myristoylation sequence, an IL-15 sequence, a linker sequence, and an IL-15Rα sequence.

In some embodiments, the fusion protein comprises an amino acid sequence set forth in any one of SEQ ID NOs: 21-24, or an amino acid sequence having at least 90%, 95%, or 99% identity to the amino acid sequence set forth in any one of SEQ ID NOs: 21-24.

In some embodiments, the fusion protein comprises an amino acid sequence set forth in SEQ ID NO: 24, or an amino acid sequence having at least 90%, 95%, or 99% identity to the amino acid sequence set forth in SEQ ID NO: 24.

In some embodiments, the fusion protein is trapped inside the cells to reduce the amount of secreted IL-15 and therefore reduce the toxicity of IL-15.

In one aspect, the present disclosure is related to a nucleic acid comprising one or more nucleic acid sequences encoding the fusion protein described herein or a portion thereof.

In some embodiments, the nucleic acid further comprises a second nucleic acid sequence encoding an engineered receptor, wherein the engineered receptor comprises an extracellular antigen binding domain or ligand binding domain, and optionally an intracellular signaling domain.

In some embodiments, the engineered receptor nucleic acid sequence and the nucleic acid sequences encoding the fusion protein are separated by a third nucleic acid sequence encoding a cleavable linker.

In some embodiments, the cleavable linker comprises an amino acid sequence set forth in SEQ ID NO: 2, or an amino acid sequence having at least 90%, 95%, or 99% identity to the amino acid sequence set forth in any one of SEQ ID NO: 2.

In some embodiments, the engineered receptor is selected from the group consisting of an engineered T cell receptor (TCR), a chimeric antigen receptor (CAR), a T cell antigen coupler (TAC) or a portion thereof.

In some embodiments, the engineered receptor is a CAR.

In some embodiments, the CAR comprises an extracellular antigen binding domain that specifically binds to an antigen, wherein the antigen is a tumor antigen selected from the group consisting of CD19, CD20, CD22, CD30, CD33, CD38, BCMA, CS1, CD138, CD123/IL3Rα, c-Met, gp100, MUC1, IGF-I receptor, EpCAM, EGFR/EGFRvIII, HER2, IGF1R, mesothelin, PSMA, WT1, ROR1, CEA, GD-2, NY-ESO-1, MAGE A3, DLL3, GPC3, guanylate cyclase 2C (GCC), Claudin18.2, Claudin6, Glycolipid F77, PD-L1, and/or PD-L2.

In some embodiments, the tumor antigen is BCMA.

In some embodiments, the CAR comprises a first VHH antibody moiety that comprises a CDR1 comprising the amino acid sequence of SEQ ID NO: 29, a CDR2 comprising the amino acid sequence of SEQ ID NO: 30, and a CDR3 comprising the amino acid sequence of SEQ ID NO: 31, and a second VHH antibody moiety that comprises a CDR1 comprising the amino acid sequence of SEQ ID NO: 32, a CDR2 comprising the amino acid sequence of SEQ ID NO:33, and a CDR3 comprising the amino acid sequence of SEQ ID NO: 34.

In some embodiments, the CAR comprises a first VHH antibody moiety comprising the amino acid sequences of SEQ ID NO: 27 or an amino acid sequence having at least 90%, 95%, or 99% identity to the amino acid sequence of SEQ ID NO: 27, and a second VHH antibody moiety comprising the amino acid sequences of SEQ ID NO: 28 or an amino acid sequence having at least 90%, 95%, or 99% identity to the amino acid sequence of SEQ ID NO: 28.

In some embodiments, the CAR comprises an amino acid sequence set forth in SEQ ID NO: 1, or an amino acid sequence having at least 90%, 95%, or 99% identity to the amino acid sequence set forth in any one of SEQ ID NO: 1.

In one aspect, the present disclosure is related to a vector comprising the nucleic acid 5 described herein.

In one aspect, the present disclosure is related to a cell comprising the fusion protein described herein, the nucleic acid described herein, and/or the vector described herein.

In some embodiments, the cell expresses an engineered receptor.

In some embodiments, the engineered receptor is selected from the group consisting of an engineered T cell receptor (TCR), a chimeric antigen receptor (CAR), a T cell antigen coupler (TAC) or a portion thereof.

In some embodiments, the engineered receptor specifically recognizes a tumor antigen.

In some embodiments, the cell an immune cell.

In some embodiments, the cell is selected from a group consisting of T cell, αβT cell, γδT cell, NK cell, peripheral blood mononuclear cell (PBMC), hematopoietic stem cell, pluripotent stem cell, an embryonic stem cell, and a combination thereof.

In some embodiments, the cell is a NK cell.

In one aspect, the present disclosure is related to a method for producing the cell described herein, the method comprising introducing the vector described herein into a cell.

In one aspect, the present disclosure is related to a method of treating a subject having cancer, the method comprising administering to the subject in need thereof a therapeutically effective amount of the cell described herein.

In some embodiments, the subject has breast cancer, lung cancer, pancreatic cancer, melanoma, oral cancer, mesothelioma, ovarian cancer, colorectal cancer, gastric cancer, cervical cancer, brain cancer, skin cancer, multiple myeloma, lymphoma, epithelial neoplasms, soft tissue sarcoma, esophageal cancers, or CNS tumors.

In one aspect, the disclosure is related to a modified IL-15 (e.g., a modified IL-15/IL-15R complex) comprising an ER retention sequence and/or a myristoylation sequence. The modified IL-15 provides an immunostimulatory/activating signal. The disclosure further relates to polynucleotides encoding the modified IL-15, modified cells expressing the modified IL-15, therapeutic use of the modified IL-15, and pharmaceutical compositions comprising the modified IL-15.

In one aspect, the disclosure relates to modified IL-15 (e.g., a modified IL-15/IL-15R complex) comprising an endoplasmic reticulum (ER) retention sequence and/or a myristoylation sequence, and methods of use thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1B show the amount of cell surface IL-15 and IL-15Rα in the cells transfected with various modified IL-15 and CAR constructs (transfected cells or modified cells). Selected construct designs were tested for IL-15 and IL-15Rα expression on Day 18 using antibodies against IL-15 and IL-15Rα. FIG. 1A shows the percentage of IL-15 and IL-15Rα positive NK cells. FIG. 1B shows the MFI of IL-15 and IL-15Rα.

FIG. 2 shows the percentage of CAR positive cells in the transfected cells. Selected construct designs were tested for CAR positive rates in the transfected cells using BCMA-FITC. All constructs showed a good and comparable expression of the BCMA CAR.

FIGS. 3A-3B show the cellular the proliferation and viability of the transfected cells. Selected construct designs were tested for cellular proliferation by cell counting and for viability with trypan blue staining. Construct 32 was used as control. Cell cultures with equal number of cells were seeded on Day 0 in cytokine free medium and were counted on Day 9. FIG. 3A shows cell numbers (millions) for each construct design. FIG. 3B shows viability for each construct design.

FIGS. 4A-4B show cellular proliferation of the transfected cells. Selected construct designs were tested for cellular proliferation using the dye dilution method. Construct 32 was used as control. Cell trace violet labeled cells in equal number were seeded on Day 0 in cytokine free medium and were evaluated for dye dilution on Day 1 and Day 9. FIG. 4A shows the decrease in MFI for each construct design. FIG. 4B shows the fold proliferation for each construct design.

FIGS. 5A-5B show the percentages of phosphorylated STAT5 (pSTAT5) positive cells in the transfected cells. Selected construct designs were tested for STAT5 activing activity by measurement of pSTAT5 using a pSTAT5 antibody. FIG. 5A shows the percentages of pSTAT5 positive cells in cells transfected with different IL-15 constructs. FIG. 5B shows the MFI for each construct. Mock data is gated on total NK cells, while the remaining data is gated on CAR positive cells.

FIG. 6 shows cytotoxicity of the transfected cells against tumor cells. Selected construct designs were tested for cytotoxicity by a serial cytotoxicity assay against H929 cells, where the transfected cells were repeatedly challenged with tumor cells. Specifically, fresh trace violet labeled H929 cells were added at the beginning of each round and live tumor cell were counted 24 hours later using flow cytometer.

FIG. 7 shows the safety of CAR constructs. Selected construct designs were tested for proliferation in absence of tumor antigen in cytokine free medium. Cell cultures with equal number of cells were seeded on Day 0 in cytokine free medium and were counted on various time points until Day 49. FIG. 7A shows cell numbers (million). FIG. 7B shows viability of the modified cells for each construct design.

FIGS. 8A-8G show cytotoxicity of the transfected cells against tumor cells in vivo. Selected construct designs were tested for activity against NCI-H929 cells in vivo. To create the tumor xenograft, NCG mice were injected intravenously with 2 million Luciferase labeled NCI-H929 tumor cells. Twelve days post-tumor engraftment, mice were injected intravenously (i.v.) with 2 million IL-15 construct armored CAR-NK cells or unNK cells. FIG. 8A shows weekly monitoring of tumor progression using in vivo bioluminescence imaging (BLI). FIG. 8B shows survival curve of mice treated with different IL-15 constructs armored BCMA CAR-NK cells. FIGS. 8C-8F show persistence and pharmacokinetics of NK cells. These figures illustrate the expansion of human CD45-positive NK cells (percentage in FIG. 8C and absolute numbers in FIG. 8E) and CAR-positive NK cells (percentage in FIG. 8D and absolute numbers in FIG. 8F) within peripheral blood at various time points. FIG. 8G shows the body weight change of the mice.

FIG. 9 shows selected sequences in the present application.

### DETAILED DESCRIPTION

IL-15 is a key cytokine exhibiting a pleiotropic effect on the development, proliferation and activation of natural killer cells, as well as the proliferation and activation in CD8+ T-cell, therefore being considered as one of most promising molecules for anti-cancer immune therapy.

IL-15 drew much attention due to its similarity to IL-2 in its cytokine receptor biology. IL-15R is a heterotrimeric receptor consisting of a unique IL-15Rα chain, a shared β subunit with IL-2 (CD122) and a common γ subunit (CD132) shared with several cytokines, implying similar biological activities of IL-2 and IL-15.

A unique aspect of IL-15 is that it employs trans-presentation in which IL-15-producing cells present the cytokine in the context of IL-15 receptor α (IL-15Rα) chain on the cell surface. A pivotal role of dendritic cell (DC)-expressed IL-15Rα for trans-presenting IL-15 to NK cells has been demonstrated. Engagement of IL-15R on NK cells causes the auto-phosphorylation and activation of Janus Kinases (JAK1 and JAK3), which induces at least three parallel signaling cascades: Ras-Raf-MAPK, signal transduction and activation of transcription (STAT) 5 and PI3K-AKT-mTOR pathways.

Upon IL-15 binding to IL-15Rβ/γ complex, a conformational change results in the phosphorylation and activation of receptor-associated JAK1 and JAK3, and subsequent tyrosine phosphorylation of IL-15Rβ/γ itself. These phospho-tyrosine residues provide binding sites for SH2-containing proteins including STAT5 molecules that are in turn phosphorylated, resulting in STAT5 dimer and/or tetramer formation and translocation to the nucleus to induce target gene expression. The expression of one such STAT5 target gene, Mcl1, is continuously required to maintain NK cell survival. Owing to its very short half-life, termination of IL-15 signaling results in the rapid loss of MCL1 in NK cells culminating in apoptosis.

Exogenous administration of IL-15 can be used to support the in vivo proliferation and anti-tumor activity of NK CAR cells, thus, overcoming the requirement to include IL-15 in the construct. However, IL-15, even when administered at a low dose of 0.5 μg/mouse every 2-3 days is associated with significant toxicity when administered in combination with CAR.

To reduce the toxicity of IL-15, modifications can be made to IL-15 to trap IL-15 inside the cells either in endoplasmic reticulum or below plasma membrane to reduce the amount of secreted IL-15 and therefore reduce the toxicity of IL-15. The present disclosure provides modified IL-15 or the related fusion proteins comprising an IL-15 sequence with reduced toxicity.

As used herein, the term “fusion protein” refers to a protein complex having one or more polypeptides with desired functions. The protein complex may comprise or consist of one polypeptide. The fusion protein may be a fusion polypeptide.

As used herein, the term “IL-15” refers to a polypeptide derived from a wild-type IL-15 or a functional variant thereof. The IL-15 may be a wild-type IL-15 (e.g., human IL-15). The IL-15 can have one or more mutations (e.g., insertions, deletions, or substitutions). The IL-15 may be human IL-15. The IL-15 sequence can be the full sequence of IL-15, or a portion of the full sequence of IL-15.

As used herein, the term “IL-15Rα” refers to a polypeptide derived from a wild-type IL-15Rα, a functional variant thereof, or a portion thereof. The IL-15Rα may be a wild-type IL-15Rα (e.g., human IL-15Rα). The IL-15Rα can have one or more mutations (e.g., insertions, deletions, or substitutions). The IL-15Rα may be human IL-15Rα. The IL-15Rα sequence can be the full sequence of IL-15Rα, or a portion of the full sequence of IL-15Rα. The IL-15Rα sequence can comprise or consist of the sequence of the sushi domain of IL-15Rα.

As used herein, a “vector” is any construct capable of delivering one or more polynucleotides of interest to a host cell when the vector is introduced to the host cell. An “expression vector” is capable of delivering and expressing the one or more polynucleotides of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

As used herein, the term “chimeric antigen receptor” or “CAR” as used herein refers to genetically engineered receptors, which can be used to graft one or more antigen specificity onto immune effector cells, such as T cells or NK cells. Some CARs are also known as “artificial T-cell receptors,” “chimeric T cell receptors,” or “chimeric immune receptors.” The CAR may comprise an extracellular ligand binding domain or an extracellular antigen binding domain specific for one or more antigens (such as tumor antigens), a transmembrane region, and an intracellular signaling domain of a T cell and/or other receptors. “CAR-T cell” refers to a T cell that expresses a CAR. “CAR-NK cell” refers to a NK cell that expresses a CAR.

As used herein, the term “T-cell receptor” or “TCR” as used herein refers to an endogenous or modified T-cell receptor comprising an extracellular antigen binding domain that binds to a specific antigenic peptide bound in an MHC molecule. The TCR may comprise a TCRα polypeptide chain and a TCRβ polypeptide chain. The TCR may comprise a TCRγ polypeptide chain and a TCRδ polypeptide chain. The TCR may specifically bind a tumor antigen. “TCR-T” refers to a T cell that expresses a recombinant TCR. Expression of a heterologous antigen receptor, such as a heterologous TCR or CAR, can alter the immunogenic specificity of the T cells so that they recognize or display improved recognition for one or more tumor antigens that are present on the surface of the cancer cells of an individual with cancer.

As used herein, the term “cancer” refers to cells having the capacity for autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, and cancer of the small intestine. Cancer that is “naturally arising” includes any cancer that is not experimentally induced by implantation of cancer cells into a subject, and includes, for example, spontaneously arising cancer, cancer caused by exposure of a subject to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation. The term “hematopoietic neoplastic disorders” includes diseases involving hyperplastic/neoplastic cells of hematopoietic origin. A hematopoietic neoplastic disorder can arise from myeloid, lymphoid or erythroid lineages, or precursor cells thereof.

As used herein, the term “subject” refers to an animal, human or non-human, to whom treatment according to the methods of the present disclosure is provided. Veterinary and non-veterinary applications are contemplated by the present disclosure. Human subjects can be adult humans or juvenile humans (e.g., humans below the age of 18 years old). In addition to humans, subjects include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### Modified IL15 and Related Fusion Proteins

The present disclosure provides modified IL-15 and related fusion proteins comprising an IL-15 sequence with reduced toxicity. The modified IL-15 and related fusion proteins may be trapped inside cells and the amount of secreted IL-15 is reduced, which can reduce the toxicity. The modified IL-15 and related fusion proteins can lead to increased persistence of NK cells in-vivo.

In one aspect, the disclosure provides modified IL-15 or the related fusion proteins that can target certain locations inside the cell. The modified IL-15 or the related fusion proteins can have a calreticulin signal peptide sequence. The modified IL-15 or the related fusion proteins can have an endoplasmic reticulum (ER) retention sequence. The modified IL-15 or the related fusion proteins can have a myristoylation sequence. The modified IL-15 or the related fusion proteins can have an IL-15Rα sequence. The modified IL-15 or the related fusion proteins may be co-expressed with an engineered receptor (e.g., CAR or TCR).

The modified IL-15 or the related fusion proteins can enhance the proliferation of NK cells. The modified IL-15 and related fusion proteins can be used to improve persistence and function of immune effector cells, such as NK, T (αβ-T and δγ-T), Treg or NKT cells. The modified IL-15 or the related fusion proteins can be expressed in genetically modified immune cells, such as CAR-T, CAR-NK, CAR-NKT cells. The modified cells (e.g., modified CAR-NK cells) can be used for treating cancers and autoimmune diseases. Other cell types, such as mesenchymal stem cells (MSC), hepatocytes in additional to immune cells can be used to express modified IL-15 or the related fusion proteins as described herein.

In one aspect, the disclosure relates to various modified IL-15 or the related fusion proteins. Various modifications can be made to the recombinant IL-15 to modify its distribution within the cell and the amount secreted. The modified IL-15 or the related fusion proteins may contain a calreticulin signal peptide sequence, which mediate the modified IL-15 or the related fusion proteins targeting to the membrane of the ER. The modified IL-15 or the related fusion proteins may contain an ER retention sequence, which helps to retain the modified IL-15 or the related fusion proteins in the ER. The modified IL-15 or the related fusion proteins may contain a myristoylation sequence, which helps to retain the modified IL-15 inside the cell. The modified IL-15 or the related fusion proteins may contain palmitoylation sequence, which helps to retain the modified IL-15 inside the cell. The modified IL-15 or the related fusion proteins may contain and prenylation sequence, which helps to retain the modified IL-15 inside the cell.

The modified IL-15 or the related fusion proteins described herein may have the structures and or sequences described in Table 1 and Table 2.

The modified IL-15 or the related fusion proteins may comprise an IL-15 sequence. The IL-15 sequence may comprise or consist of an amino acid sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 6. The IL-15 sequence can have 1, 2, 3, 4, 5, 6, 7, 8, 9, 10 or more than 10 mutations (e.g., as compared to SEQ ID NO: 6 or the entire wild-type IL-15). These mutations can be amino acid insertions, deletions, or substitutions. The insertions, deletions, and substitutions can be within the IL-15 sequence, and/or at one or both terminal ends of the IL-15 sequence.

The modified IL-15 or the related fusion proteins may comprise an IL-15 propeptide sequence. The IL-15 propeptide sequence may comprise or consist of an amino acid sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 5. The IL-15 propeptide sequence can have 1, 2, 3, 4, 5, 6, 7, 8, 9, 10 or more than 10 mutations (e.g., as compared to SEQ ID NO: 5). These mutations can be amino acid insertions, deletions, or substitutions. The insertions, deletions, and substitutions can be within the IL-15 propeptide sequence, and/or at one or both terminal ends of the IL-15 propeptide sequence.

The modified IL-15 or the related fusion proteins may comprise a signal peptide sequence. The signal peptide sequence may comprise or consist of an amino acid sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 3 or SEQ ID NO: 4. The signal peptide sequence can have 1, 2, 3, 4, 5, 6, 7, 8, 9, 10 or more than 10 mutations (e.g., as compared to SEQ ID NO: 3 or SEQ ID NO: 4). These mutations can be amino acid insertions, deletions, or substitutions. The insertions, deletions, and substitutions can be within the signal peptide sequence, and/or at one or both terminal ends of the signal peptide sequence.

The modified IL-15 or the related fusion proteins may comprise an ER retention sequence. The ER retention sequence may comprise or consist of an amino acid sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 12 or SEQ ID NO: 13. The ER retention sequence can have 1, 2, 3, 4, 5, 6, 7, 8, 9, 10 or more than 10 mutations (e.g., as compared to SEQ ID NO: 12 or SEQ ID NO: 13). These mutations can be amino acid insertions, deletions, or substitutions. The insertions, deletions, and substitutions can be within the ER retention sequence, and/or at one or both terminal ends of the ER retention sequence.

The modified IL-15 or the related fusion proteins may comprise an IL-15Rα sequence. The IL-15Rα sequence may comprise or consist of an amino acid sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 11. The IL-15Rα sequence can have 1, 2, 3, 4, 5, 6, 7, 8, 9, 10 or more than 10 mutations (e.g., as compared to SEQ ID NO: 11). These mutations can be amino acid insertions, deletions, or substitutions. The insertions, deletions, and substitutions can be within the IL-15Rα sequence, and/or at one or both terminal ends of the IL-15Rα sequence.

IL-15Rα Sushi domain is a region that results in high-affinity binding of trans-presented IL-15 to IL-15Rβ/γ. The modified IL-15 or the related fusion proteins may comprise an IL-15Rα sequence, wherein the IL-15Rα sequence comprises or consists of an IL-15Rα Sushi domain sequence. The IL-15Rα Sushi domain sequence may comprise or consist of an amino acid sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 10. The IL-15Rα Sushi domain sequence can have 1, 2, 3, 4, 5, 6, 7, 8, 9, 10 or more than 10 mutations (e.g., as compared to SEQ ID NO: 10). These mutations can be amino acid insertions, deletions, or substitutions. The insertions, deletions, and substitutions can be within the IL-15Rα Sushi domain sequence, and/or at one or both terminal ends of the IL-15Rα Sushi domain sequence.

The modified IL-15 or the related fusion proteins may comprise a myristoylation sequence. The myristoylation sequence may comprise an amino acid sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of any one of SEQ ID NOs: 14-16. The myristoylation sequence can have 1, 2, 3, 4, 5, 6, 7, 8, 9, 10 or more than 10 mutations (e.g., as compared to any one of SEQ ID NOs: 14-16). These mutations can be amino acid insertions, deletions, or substitutions. The insertions, deletions, and substitutions can be within the myristoylation sequence, and/or at one or both terminal ends of the myristoylation sequence.

The modified IL-15 or the related fusion proteins may comprise a linker sequence. The linker sequence may comprise or consist of an amino acid sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of any one of SEQ ID NOs: 7-9. The linker sequence can have 1, 2, 3, 4, 5, 6, 7, 8, 9, 10 or more than 10 mutations (e.g., as compared to any one of SEQ ID NOs: 7-9). These mutations can be amino acid insertions, deletions, or substitutions. The insertions, deletions, and substitutions can be within the linker sequence, and/or at one or both terminal ends of the linker sequence.

The modified IL-15 or the related fusion proteins may comprise or consist of, from N-terminus to C-terminus, a signal peptide sequence (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 3 or SEQ ID NO: 4), a linker sequence (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOs: 7-9), an IL-15 sequence (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 6), a linker sequence (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOs: 7-9), and an ER retention sequence (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 12 or SEQ ID NO: 13). The modified IL-15 or the related fusion proteins may comprise an amino acid sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 17. The modified IL-15 or the related fusion proteins can have 1, 2, 3, 4, 5, 6, 7, 8, 9, 10 or more than 10 mutations (e.g., as compared to SEQ ID NO: 17). These mutations can be amino acid insertions, deletions, or substitutions. The insertions, deletions, and substitutions can be within the modified IL-15 or the related fusion proteins, and/or at one or both terminal ends of the modified IL-15 or the related fusion proteins.

The modified IL-15 or the related fusion proteins may comprise or consist of, from N-terminus to C-terminus, a signal peptide sequence (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 3 or SEQ ID NO: 4), an IL-15 sequence (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 6), a linker sequence (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOs: 7-9), an IL-15Rα sequence (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 10 or SEQ ID NO: 11), and an ER retention sequence (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 12 or SEQ ID NO: 13). The modified IL-15 or the related fusion proteins may comprise an amino acid sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 18. The modified IL-15 or the related fusion proteins can have 1, 2, 3, 4, 5, 6, 7, 8, 9, 10 or more than 10 mutations (e.g., as compared to SEQ ID NO: 18). These mutations can be amino acid insertions, deletions, or substitutions. The insertions, deletions, and substitutions can be within the modified IL-15 or the related fusion proteins, and/or at one or both terminal ends of the modified IL-15 or the related fusion proteins.

The modified IL-15 or the related fusion proteins may comprise or consist of, from N-terminus to C-terminus, a signal peptide (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 3 or SEQ ID NO: 4), a linker sequence (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOs: 7-9), an IL-15 sequence (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 6), a linker sequence (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOs: 7-9), an IL-15Rα sequence (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 10 or SEQ ID NO: 11), and an ER retention sequence (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 12 or SEQ ID NO: 13). The modified IL-15 or the related fusion proteins may comprise an amino acid sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 19. The modified IL-15 or the related fusion proteins can have 1, 2, 3, 4, 5, 6, 7, 8, 9, 10 or more than 10 mutations (e.g., as compared to SEQ ID NO: 19). These mutations can be amino acid insertions, deletions, or substitutions. The insertions, deletions, and substitutions can be within the modified IL-15 or the related fusion proteins, and/or at one or both terminal ends of the modified IL-15 or the related fusion proteins.

The modified IL-15 or the related fusion proteins may comprise or consist of, from N-terminus to C-terminus, a signal peptide (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 3 or SEQ ID NO: 4), a linker sequence (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOs: 7-9), an IL-15 sequence (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 6), a linker sequence (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOs: 7-9), an IL-15Rα sequence (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 10 or SEQ ID NO: 11), and an ER retention sequence (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 12 or SEQ ID NO: 13). The modified IL-15 or the related fusion proteins may comprise an amino acid sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 20. The modified IL-15 or the related fusion proteins can have 1, 2, 3, 4, 5, 6, 7, 8, 9, 10 or more than 10 mutations (e.g., as compared to SEQ ID NO: 20). These mutations can be amino acid insertions, deletions, or substitutions. The insertions, deletions, and substitutions can be within the modified IL-15 or the related fusion proteins, and/or at one or both terminal ends of the modified IL-15 or the related fusion proteins.

The modified IL-15 or the related fusion proteins may comprise or consist of, from N-terminus to C-terminus, a signal peptide (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 3 or SEQ ID NO: 4), a myristoylation sequence (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOs: 14-16), an IL-15 sequence (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 6), a linker sequence (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOs: 7-9), and an IL-15Rα sequence (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 10 or SEQ ID NO: 11). The modified IL-15 or the related fusion proteins may comprise an amino acid sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 21. The modified IL-15 or the related fusion proteins can have 1, 2, 3, 4, 5, 6, 7, 8, 9, 10 or more than 10 mutations (e.g., as compared to SEQ ID NO: 21). These mutations can be amino acid insertions, deletions, or substitutions. The insertions, deletions, and substitutions can be within the modified IL-15 or the related fusion proteins, and/or at one or both terminal ends of the modified IL-15 or the related fusion proteins.

The modified IL-15 or the related fusion proteins may comprise or consist of, from N-terminus to C-terminus, a myristoylation sequence (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOs: 14-16), an IL-15 sequence (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 6), a linker sequence (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOs: 7-9), and an IL-15Rα sequence (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 10 or SEQ ID NO: 11). The modified IL-15 or the related fusion proteins may comprise an amino acid sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 22. The modified IL-15 or the related fusion proteins can have 1, 2, 3, 4, 5, 6, 7, 8, 9, 10 or more than 10 mutations (e.g., as compared to SEQ ID NO: 22). These mutations can be amino acid insertions, deletions, or substitutions. The insertions, deletions, and substitutions can be within the modified IL-15 or the related fusion proteins, and/or at one or both terminal ends of the modified IL-15 or the related fusion proteins.

The modified IL-15 or the related fusion proteins may comprise or consist of, from N-terminus to C-terminus, a signal peptide (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 3 or SEQ ID NO: 4), a myristoylation sequence (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOs: 14-16), an IL-15 sequence (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 6), a linker sequence (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOs: 7-9), and an IL-15Rα sequence (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 10 or SEQ ID NO: 11). The modified IL-15 or the related fusion proteins may comprise an amino acid sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 23. The modified IL-15 or the related fusion proteins can have 1, 2, 3, 4, 5, 6, 7, 8, 9, 10 or more than 10 mutations (e.g., as compared to SEQ ID NO: 23). These mutations can be amino acid insertions, deletions, or substitutions. The insertions, deletions, and substitutions can be within the modified IL-15 or the related fusion proteins, and/or at one or both terminal ends of the modified IL-15 or the related fusion proteins.

The modified IL-15 or the related fusion proteins may comprise or consist of, from N-terminus to C-terminus, a signal peptide (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 3 or SEQ ID NO: 4), a myristoylation sequence (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOs: 14-16), an IL-15 sequence (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 6), a linker sequence (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOs: 7-9), and an IL-15Rα sequence (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 10 or SEQ ID NO: 11). The modified IL-15 or the related fusion proteins may comprise an amino acid sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 24. The modified IL-15 or the related fusion proteins can have 1, 2, 3, 4, 5, 6, 7, 8, 9, 10 or more than 10 mutations (e.g., as compared to SEQ ID NO: 24). These mutations can be amino acid insertions, deletions, or substitutions. The insertions, deletions, and substitutions can be within the modified IL-15 or the related fusion proteins, and/or at one or both terminal ends of the modified IL-15 or the related fusion proteins.

The modified IL-15 or the related fusion proteins may comprise or consist of, from N-terminus to C-terminus, an IL-15 sequence (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 6). The modified IL-15 or the related fusion proteins may comprise an amino acid sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 25. The modified IL-15 or the related fusion proteins can have 1, 2, 3, 4, 5, 6, 7, 8, 9, 10 or more than 10 mutations (e.g., as compared to SEQ ID NO: 25). These mutations can be amino acid insertions, deletions, or substitutions. The insertions, deletions, and substitutions can be within the modified IL-15 or the related fusion proteins, and/or at one or both terminal ends of the modified IL-15 or the related fusion proteins.

The modified IL-15 or the related fusion proteins may comprise or consist of, from N-terminus to C-terminus, a signal peptide (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 3 or SEQ ID NO: 4), an IL-15 propeptide sequence (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 5), and an IL-15 sequence (e.g., a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 6). The modified IL-15 or the related fusion proteins may comprise an amino acid sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 26. The modified IL-15 or the related fusion proteins can have 1, 2, 3, 4, 5, 6, 7, 8, 9, 10 or more than 10 mutations (e.g., as compared to SEQ ID NO: 26). These mutations can be amino acid insertions, deletions, or substitutions. The insertions, deletions, and substitutions can be within the modified IL-15 or the related fusion proteins, and/or at one or both terminal ends of the modified IL-15 or the related fusion proteins.

The modified IL-15 or the related fusion proteins may be trapped in the ER of the cells. The modified IL-15 or the related fusion proteins may be trapped inside the cells (e.g., cell plasma).

### NK Cell Therapy

Chimeric antigen receptors (CARs) that redirect the specificity of autologous T-cells against lymphoid malignancies have produced striking clinical results. Nonetheless, CAR-modified T-cells have a number of limitations. The generation of an autologous product for each individual subject is logistically cumbersome and restrictive for widespread clinical use. The manufacturing of CAR T-cells often takes several weeks, making it impractical for subjects with rapidly advancing disease. Furthermore, it is not always possible to generate clinically relevant doses of CAR T-cells from heavily pre-treated, often lymphopenic subjects. A previously collected allogeneic product could overcome these limitations; however, allogeneic T-cells (even if HLA-matched) carry a risk of graft-versus-host disease (GVHD), mediated through their native αβ T-cell receptor.

Natural killer (NK) cells provide an attractive alternative to T-cells for CAR engineering. NK cells do not cause GVHD, and thus open opportunities to produce an off-the-shelf product for immediate clinical use. Moreover, as engineered NK cells should also retain their full array of native receptors, they have the potential to exert cytotoxicity through mechanisms other than that dictated by the specificity of the CAR, which in principle could reduce the risk of relapse mediated by loss of CAR-targeted antigen, as reported for CAR-T cell therapy.

Functional NK cells can be derived from several sources. Autologous NK cells can be reproducibly generated in vitro. Cord blood (CB) is a readily available source of allogeneic NK cells with clear advantages. CB is available as an off-the-shelf frozen product, an advantage that has been bolstered by methods to generate large numbers of highly functional NK cells from frozen CB units ex vivo. The generation of CAR-transduced NK cells from frozen CB units stored in large global CB bank inventories holds promise for widespread scalability that cannot be replicated with individual adult donors who require screening and leukapheresis. However, a major disadvantage of NK cells is their lack of persistence after adoptive transfer in the absence of cytokine support. Finally, CAR-engineered NK cells may also exert potentially serious toxicity, such as cytokine release syndrome (CRS) or off-tumor/on-target toxicity, as reported with CAR T-cells.

Mature NK cells have a short lifespan with poor in vivo persistence both in humans and in mice. Although recent data support the existence of long-lived memory NK cells in mice and possibly in humans, the absence of a reliable and stable marker (or set of markers) to define memory NK cells hinders their selection for immunotherapy. This poses a major limitation on their use for adoptive therapy, as in vivo persistence of effector cells is crucial for sustained clinical responses. IL-15 is a cytokine that drives NK cell expansion and persistence. Ectopic production of IL-15 can lead to more robust activation of NK cells with enhanced in vivo proliferation, persistence and anti-tumor activity than that seen with CAR-transduced NK cells lacking IL-15. Although the latter could mediate an antitumor response, the effect was only transient, further emphasizing the importance of in vivo persistence of CAR-expressing NK cells for effective and durable antitumor immunity.

In cancer subjects, NK cells frequently display an impaired function. Thus, primary strategies in immunotherapy are aimed to boost in vivo NK cell-mediated antitumor activity. One approach is based on the in vivo administration of cytokines, such as IL-2 and IL-15, that determine NK cell activation, differentiation, and expansion. IL-2 administration was approved in the 1990s for the treatment of metastatic RCC and melanoma subjects. Two major obstacles in IL-2-based therapy are the dose-associated toxicity (primarily vascular leakage) and the induction of T regulatory (Treg) cell activation and expansion, thus resulting in inhibition of NK cell function. Recently, IL-2 variants, with lower affinity for IL-2Rα subunit (highly expressed by Treg cells), have been designed. In addition, PEGylated IL-2 (also known as NKTR-214) that binds CD122 (IL-2Rβ), expressed by both T and NK cells, is able to boost preferentially these cells and their anti-tumor responses.

### Signal Peptide (SP)

Cleavable endoplasmic reticulum (ER) signal peptides (SPs) and other non-cleavable signal sequences target roughly a quarter of the human proteome to the ER. These short peptides, mostly located at the N-termini of proteins, are highly diverse. For most proteins targeted to the ER, it is the interactions between the signal sequences and the various ER targeting and translocation machineries such as the signal recognition particle (SRP), the protein-conducting channel Sec61, and the signal peptidase complex (SPC) that determine the proteins' target location and provide translocation fidelity.

The secretory pathway is a protein trafficking highway utilized by more than a quarter of the human proteome. Soluble secreted proteins such as antibodies and protein hormones rely on this pathway. The pathway also delivers transmembrane proteins (TMPs) to the endoplasmic reticulum (ER), its downstream organelles such as the Golgi apparatus, and the plasma membrane.

All secretory proteins are translated by cytosolic ribosomes and must be first targeted to and then transported across (or inserted into) the ER membrane at the early stage of their life, either co- or post-translationally. A complex network of cytosolic and ER membrane-resident macromolecules facilitate and assist the ER targeting and translocation.

SPs are characterized by a tripartite structure: (i) an often positively charged, N-terminal ‘n-region’ that faces the cytosol; (ii) a short hydrophobic core-most commonly between 7 and 15, but not longer than 18-20 amino acids called ‘h-region’; and (iii) a polar luminal C-terminal ‘c-region’ that contains the scissile bond and must be occupied by short, hydrophobic residues at positions-1 and -3 relative to the cleavage site. Initially, SPs are inserted into the ER membrane with the N-terminus facing towards the cytosol (Nin) and the mature sequence facing the organellar lumen (Cout).

The disclosure provides a signal peptide sequence. The signal peptide sequence may comprise an amino acid sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 3 or SEQ ID NO: 4. The signal peptide sequence can have 1, 2, 3, 4, 5, 6, 7, 8, 9, 10 or more than 10 mutations (e.g., as compared to SEQ ID NO: 3 or SEQ ID NO: 4). These mutations can be amino acid insertions, deletions, or substitutions. The insertions, deletions, and substitutions can be within the signal peptide sequence, and/or at one or both terminal ends of the signal peptide sequence.

The modified IL-15 or the related fusion proteins may contain a signal peptide sequence.

Calreticulin (CALR) is a 46 kDa ER luminal Ca2+-binding protein and molecular chaperone. The protein contains an N-terminal cleavable signal peptide sequence that directs it to the ER.

The signal peptide sequence may be calreticulin signal peptide (CALRSP) sequence. The calreticulin signal peptide sequence may comprise an amino acid sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 3. The signal peptide sequence can have 1, 2, 3, 4, 5, 6, 7, 8, 9, 10 or more than 10 mutations (e.g., as compared to SEQ ID NO: 3). These mutations can be amino acid insertions, deletions, or substitutions. The insertions, deletions, and substitutions can be within the signal peptide sequence, and/or at one or both terminal ends of the signal peptide sequence.

The modified IL-15 or the related fusion proteins may contain a calreticulin signal peptide sequence. The calreticulin signal peptide sequence combined with an ER retention sequence can help to retain the modified IL-15 or the related fusion proteins in the ER more efficiently than other signal peptides.

### ER Retention Sequence

After their co-translational insertion into the ER lumen or the ER membrane, most proteins are transported via the Golgi apparatus downstream on the secretory pathway while a few protein species are retained in the ER. Polypeptide retention in the ER is either signal-independent or depends on specific retention signals encoded by the primary sequence of the polypeptide. A first category, i.e. the newly synthesized polypeptides that are unable to reach their final conformation, are retained in the ER where this quality control generally results in their degradation. A second category, namely the ER-resident proteins escape the bulk flow of secretion due to the presence of a specific N- or C-terminal signal that interacts with integral membrane or soluble receptors. ER retention of soluble proteins mediated by either KDEL, HDEL or related sequences.

In order to remain in the ER, and therefore diverted from the bulk flow of secretory proteins, ER-resident proteins generally require specific signals for retention or retrieval. By comparing the polypeptide sequences of many soluble proteins that reside in the ER lumen, a consensus tetrapeptide H/KDEL has been identified at their C-terminal end. Also, a carboxy-terminal tetrapeptide KDEL has been shown to function as an ER retention signal in animal cells. Many laboratories have shown that the addition of KDEL to the C-terminal end of various secreted proteins leads to the retention of these proteins in the ER of animal cells or at least to a significant retardation of their transport downstream the secretory pathway.

The disclosure provides an ER retention sequence. The ER retention sequence may comprise an amino acid sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 12 or SEQ ID NO: 13. The ER retention sequence can have 1, 2, 3, 4, 5, 6, 7, 8, 9, 10 or more than 10 mutations (e.g., as compared to SEQ ID NO: 12 or SEQ ID NO: 13). These mutations can be amino acid insertions, deletions, or substitutions. The insertions, deletions, and substitutions can be within the ER retention sequence, and/or at one or both terminal ends of the ER retention sequence.

The modified IL-15 or the related fusion proteins may contain an ER retention sequence, which helps to retain the modified IL-15 or the related fusion proteins in the ER. The modified IL-15 or the related fusion proteins may contain a calreticulin signal peptide sequence and a KDEL, which helps to retain the modified IL-15 or the related fusion proteins in the ER more efficiently than other signal peptides and ER retention sequences.

### Membrane Targeting Sequences

Membrane targeting sequences provide for transport of the fusion protein to the cell surface membrane, where the some or other sequences can encode binding of the fusion protein to the cell surface membrane. Such sequences include, but are not limited to myristoylation-targeting sequence, palmitoylation targeting sequence, prenylation sequences (i.e., farnesylation, geranyl-geranylation, CAAX Box).

The modified IL-15 or the related fusion proteins may contain a cell membrane targeting sequences, which helps to retain the modified IL-15 or the related fusion protein inside the cell. The cell membrane targeting sequence can be selected from the group consisting of myristoylation sequence, palmitoylation sequence, and prenylation sequence.

### Myristoylation Sequence

Covalent attachment of fatty acids to proteins is now a widely recognized form of protein modification. Many fatty acylated proteins play key roles in regulating cellular structure and function. The two most common forms of protein fatty acylation are modification with myristate, a 14-carbon saturated fatty acid, and palmitate, a 16-carbon saturated fatty acid. The enzymology of the myristoylation reaction is well understood. Proteins that are destined to become myristoylated begin with the sequence: Met-Gly. The initiating methionine is removed cotranslationally by methionine amino-peptidase, and myristate is linked to Gly-2 via an amide bond. N-myristoylation is catalyzed by N-myristoyl transferase.

Myristoylation tags either the N-terminal alpha groups of cysteine or glycine residues through amide bonds or lysine and cysteine side chains directly or indirectly via glycerol thioester and ester linkages. Before transfer to proteins, myristate must be activated into myristoyl coenzyme A in eukaryotes or, in bacteria, to derivatives like phosphatidylethanolamine. Myristate usually serves as a molecular anchor, allowing tagged proteins to be targeted to membranes and travel across endomembrane networks in eukaryotes.

N-myristoyl transferase (NMT) is a 50-60 kDa monomeric enzyme that catalyzes transfer of myristate from myristoyl-CoA to suitable peptide and protein substrates. To date, nearly one dozen NMTs from fungal and mammalian sources have been identified. Studies of NMT from the yeast S. cerevisiae reveal that the catalytic cycle exhibits the following Bi Bi reaction mechanism: (1) myristoyl CoA binds to NMT; (2) the peptide substrate binds to NMT; (3) myristate is transferred to the N-terminal glycine of the peptide; (4) CoA is released from the enzyme; (5) myristoyl-peptide is released. In cells, N-myristoylation is a cotranslational process that occurs while the nascent polypeptide chain is still attached to the ribosome.

The consensus sequence for NMT protein substrates is: Met-Gly-X-X-X-Ser/Thr-. The initiating Met is removed by methionine amino peptidase during translation and Gly-2 becomes the N-terminal amino acid. The requirement for Gly at the N-terminus is absolute; no other amino acid will substitute. However, not all proteins with an N-terminal glycine are N-myristoylated and the ability to be recognized by NMT depends on the downstream amino acid sequence. In general, serine or threonine is preferred at position 6 and lysine or arginine is preferred at positions 7 and/or 8.

The disclosure provides a myristoylation sequence. The myristoylation sequence may comprise an amino acid sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of any one of SEQ ID NOs: 14-16. The myristoylation sequence can have 1, 2, 3, 4, 5, 6, 7, 8, 9, 10 or more than 10 mutations (e.g., as compared to any one of SEQ ID NOs: 14-16). These mutations can be amino acid insertions, deletions, or substitutions. The insertions, deletions, and substitutions can be within the myristoylation sequence, and/or at one or both terminal ends of the myristoylation sequence.

For some proteins, the myristate moiety provides not only membrane binding, but also membrane targeting functions. For example, when Src or Gag N-terminal sequences are attached to soluble proteins, the chimeras display specific localization to the plasma membrane. Conversely, mutation of N-myristoylation sites in the yeast Gpa 1p protein or in HIV-1 Gag redirect the proteins to intracellular membranes, implying that myristate participates in plasma membrane targeting. In Mason-Pfizer monkey virus, virion cores are assembled in the cytosol and myristoylation of Gag is required for intracellular transport to the plasma membrane.

The modified IL-15 or the related fusion proteins may contain a myristoylation sequence that binding to the cell surface membrane, which helps to retain the modified IL-15 or the related fusion protein inside the cell.

### Palmitoylation Sequence

Palmitoylation is the covalent attachment of fatty acids, such as palmitic acid, to cysteine and less frequently to serine and threonine residues of proteins, which are typically membrane proteins. The precise function of palmitoylation depends on the particular protein being modified. Palmitoylation enhances the hydrophobicity of proteins and contributes to their membrane association.

The modified IL-15 or the related fusion proteins may contain a palmitoylation sequence that binding to the cell surface membrane, which helps to retain the modified IL-15 or the related fusion protein inside the cell.

### Prenylation Sequence

Prenylation is the covalent attachment of a lipid consisting of either three (farnesyl) or four (geranylgeranyl) isoprene units to a free thiol of a cysteine side chain at or near the C-terminus of a protein. Protein prenylation leads to an increased hydrophobicity of proteins, typically resulting in an increased affinity for membranes.

The modified IL-15 or the related fusion proteins may contain a prenylation sequence that binding to the cell surface membrane, which helps to retain the modified IL-15 or the related fusion protein inside the cell.

### Engineered Receptor (e.g., CAR and TCR)

The present disclosure provides cells (e.g., immune cells) that contain an engineered receptor and the modified IL-15 or the related fusion proteins. The engineered receptor may comprise an extracellular ligand binding domain or an extracellular antigen binding domain, and optionally an intracellular signaling domain. Exemplary engineered receptor includes, but are not limited to, CAR, engineered TCR, and TAC receptors. The engineered receptor may comprise an extracellular domain comprising an antigen binding domain that specifically binds to an antigen (e.g., a tumor antigen), a transmembrane region, and an intracellular signaling domain. The intracellular signaling domain may comprise a primary intracellular signaling domain and/or a co-stimulatory signaling domain. The intracellular signaling domain may comprise an intracellular signaling domain of a TCR co-receptor. The engineered receptor may be encoded by a heterologous polynucleotide operably linked to a promoter (such as a constitutive promoter or an inducible promoter).

The engineered receptor may comprise one or more specific binding domains that target at least one tumor antigen, and one or more intracellular effector domains, such as one or more primary intracellular signaling domains and/or co-stimulatory signaling domains.

The engineered receptor may be a chimeric antigen receptor (CAR). Many chimeric antigen receptors are known in the art and can be suitable for the modified cells comprising the modified IL-15 or the related fusion proteins described herein. CARs can also be constructed with a specificity for any cell surface marker by utilizing antigen binding fragments or antibody variable domains of, for example, antibody molecules.

CARs of the present disclosure comprise an extracellular domain comprising at least one antigen binding domain that specifically binds at least one tumor antigen, a transmembrane region, and an intracellular signaling domain. The intracellular signaling domain may generate a signal that promotes an immune effector function of the CAR-containing cell, e.g., a CAR-T cell. “Immune effector function or immune effector response” refers to function or response, e.g., of an immune effector cell, that enhances or promotes an immune attack of a target cell. For example, an immune effector function or response can refer to a property of a T or NK cell that promotes killing or the inhibition of growth or proliferation, of a target cell. Examples of immune effector function, e.g., in a CAR-T cell, include cytolytic activity (such as antibody-dependent cellular toxicity, or ADCC) and helper activity (such as the secretion of cytokines). The CAR may have an intracellular signaling domain with an attenuated immune effector function. The CAR may have an intracellular signaling domain having no more than about any of 90%, 80%, 70%, 60%, 50%, 40%, 30%, 20%, 10% or less of an immune effector function (such as cytolytic function against target cells) compared to a CAR having a full-length and wild-type CD3 and optionally one or more co-stimulatory signaling domains. The intracellular signaling domain may generate a signal that promotes proliferation and/or survival of the CAR containing cell. The CAR may comprise one or more intracellular signaling domains selected from the signaling domains of CD28, CD137, CD3, CD27, CD40, ICOS, GITR, and OX40. The signaling domain of a naturally occurring molecule can comprise the entire intracellular or cytoplasmic portion, or the entire native intracellular signaling domain, of the molecule, or a fragment or derivative thereof.

The intracellular signaling domain of a CAR may comprise a primary intracellular signaling domain. “Primary intracellular signaling domain” refers to cytoplasmic signaling sequence that acts in a stimulatory manner to induce immune effector functions. The primary intracellular signaling domain may contain a signaling motif known as Immunoreceptor Tyrosine-based Activation Motif, or ITAM. The primary intracellular signaling domain may comprise a functional signaling domain of a protein selected from the group consisting of CD3 zeta, CD3 gamma, CD3 delta, CD3 epsilon, common FcR gamma (FCER1G), FcR beta (Fc Epsilon Rib), CD79a, CD79b, Fcgamma R IIa, DAP10, and DAP 12. The primary intracellular signaling domain may comprise a nonfunctional or attenuated signaling domain of a protein selected from the group consisting of CD3 zeta, CD3 gamma, CD3 delta, CD3 epsilon, common FcR gamma (FCER1G), FcR beta (Fc Epsilon Rib), CD79a, CD79b, Fcgamma R IIa, DAP10, and DAP 12. The nonfunctional or attenuated signaling domain can be a mutant signaling domain having a point mutation, insertion or deletion that attenuates or abolishes one or more immune effector functions, such as cytolytic activity or helper activity, including antibody-dependent cellular toxicity (ADCC). The CAR may comprise a nonfunctional or attenuated CD3 zeta (i.e. CD3ζ or CD3z) signaling domain. The intracellular signaling domain may lack a primary intracellular signaling domain. An attenuated primary intracellular signaling domain can induce no more than about any of 90%, 80%, 70%, 60%, 50%, 40%, 30%, 20%, 10% or less of an immune effector function (such as cytolytic function against target cells) compared to CARs having the same construct, but with the wild-type primary intracellular signaling domain.

The intracellular signaling domain of a CAR may comprise one or more (such as any of 1, 2, 3, or more) co-stimulatory signaling domains. “Co-stimulatory signaling domain” can be the intracellular portion of a co-stimulatory molecule. The term “co-stimulatory molecule” refers to a cognate binding partner on an immune cell (such as T cell) that specifically binds with a co-stimulatory ligand, thereby mediating a co-stimulatory response by the immune cell, such as, but not limited to, proliferation and survival. Co-stimulatory molecules are cell surface molecules other than antigen receptors or their ligands that contribute to an efficient immune response. A co-stimulatory molecule can be represented in the following protein families: TNF receptor proteins, Immunoglobulin-like proteins, cytokine receptors, integrins, signaling lymphocytic activation molecules (SLAM proteins), and activating NK cell receptors. Co-stimulatory molecules include, but are not limited to an MHC class I molecule, BTLA and a Toll ligand receptor, as well as OX40, CD27, CD28, CDS, ICAM-1, LFA-1 (CD11a/CD18), ICOS (CD278), and 4-1BB (CD137). Further examples of such co-stimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAMI (CD226), SLAMF4 (CD244, 2B4), CD84, CD96 (Tactile), CEACAM1, CRTAM, Ly9 (CD229), CD160 (BY55), PSGL1, CDIOO (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

The CAR may comprise a single co-stimulatory signaling domain. The CAR may comprise two or more co-stimulatory signaling domains. The intracellular signaling domain may comprise a functional primary intracellular signaling domain and one or more co-stimulatory signaling domains. The CAR may lack a functional primary intracellular signaling domain (such as CD32). The CAR may comprise an intracellular signaling domain consisting of or consisting essentially of one or more co-stimulatory signaling domains. The CAR may comprise an intracellular signaling domain consisting of or consisting essentially of a nonfunctional or attenuated primary intracellular signaling domain (such as a mutant CD32) and one or more co-stimulatory signaling domains. Upon binding of the antigen binding domain to tumor antigen, the co-stimulatory signaling domains of the CAR can transduce signals for enhanced proliferation, survival and differentiation of the modified immune cells having the CAR (such as T cells), and inhibit activation induced cell death. The one or more co-stimulatory signaling domains may be derived from one or more molecules selected from the group consisting of CD27, CD28, 4-1BB (i.e., CD137), OX40, CD30, CD40, CD3, lymphocyte function-associated antigen-1 (LFA-1), CD2, CD7, LIGHT, NKG2C, B7-H3 and ligands that specially bind to CD83.

The intracellular signaling domain of a CAR may comprise a co-stimulatory signaling domain derived from CD28. The intracellular signaling domain may comprise a cytoplasmic signaling domain of CD35 and a co-stimulatory signaling domain of CD28. The intracellular signaling domain in the chimeric receptor of the present application may comprise a co-stimulatory signaling domain derived from 4-1BB (i.e., CD137). The intracellular signaling domain may comprise a cytoplasmic signaling domain of CD32 and a co-stimulatory signaling domain of 4-1BB.

The intracellular signaling domain of the CAR may comprise a co-stimulatory signaling domain of CD28 and a co-stimulatory signaling domain of 4-1BB. The intracellular signaling domain may comprise a cytoplasmic signaling domain of CD35, a co-stimulatory signaling domain of CD28, and a co-stimulatory signaling domain of 4-1BB. The intracellular signaling domain may comprise a polypeptide comprising from the N-terminus to the C-terminus: a co-stimulatory signaling domain of CD28, a co-stimulatory signaling domain of 4-1BB, and a cytoplasmic signaling domain of CD32.

The antigen binding domain of a CAR may be an antibody or an antibody fragment, such as an scFv, a Fv, a Fab, a (Fab′) 2, a single domain antibody (sdAb), or a VHH domain. The antigen binding domain of a CAR may comprise a ligand or an extracellular portion of a receptor that specifically binds to a tumor antigen. The CAR may be a monospecific, bispecific or multispecific CAR. The antigen binding domain of a CAR specifically may bind a single tumor antigen. The antigen binding domain of a CAR may bind two or more tumor antigens.

The tumor antigen may be selected from the group consisting of CD19, BCMA, NY-ESO-1, VEGFR2, MAGE-A3, VEGFR2, MAGE-A3, CD20, CD22, CD30, CD33, CD38, CEA, CS1, CD138, CD123/IL3Rα, c-Met, gp100, MUC1, IGF-I receptor, EpCAM, CEA, EGFR (such as EGFRvIII), GD2, HER2, IGF1R, mesothelin, PSMA, ROR1, WT1, Glypican 3 (GPC3), Guanylate cyclase 2C (GCC), DLL3, Claudin18.2, Claudin6, Glycolipid F77, PD-L1, PD-L2, and other tumor antigens with clinical significance, and combinations thereof. The tumor antigen may be derived from an intracellular protein of tumor cells. The tumor antigen may be expressed on the surface of tumor cells. Many TCRs specific for tumor antigens (including tumor-associated antigens) have been described, including, for example, NY-ESO-1 cancer-testis antigen, the p53 tumor suppressor antigens, TCRs for tumor antigens in melanoma (e.g., MARTI, gp 100), leukemia (e.g., WT1, minor histocompatibility antigens), and breast cancer (e.g., HER2, NY-BR1).

Many CARs targeting different tumor antigens have been widely disclosed in the field, such as CD19 CARs or BCMA CARs. The extracellular antigen-binding domain of CD19 CARs can be or include the CD19 binding fragment (e.g., FMC63, SJ25C1, or those disclosed in different patents such as WO 2022/012683, etc). BCMA CARs also have been well described, related patents include but not limited to WO 2016/014789, WO 2016/014565, WO 2013/154760, WO 2018/028647, and WO 2021/121228, etc.

The extracellular antigen-binding domain of BCMA (B-cell maturation antigen) CARs may be or include BCMA binding fragment. The BCMA binding fragment may bind to one or more epitopes on BCMA. The BCMA CARs may be bivalent CARs comprising two anti-BCMA sdAbs targeting different BCMA epitopes.

The transmembrane region of a CAR may comprise a transmembrane region chosen from the transmembrane region of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL-2R beta, IL-2R gamma, IL-7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAMI (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C. The transmembrane region of the CAR may be a CD4, CD3, CD8α, or CD28 transmembrane region. The transmembrane region of the CAR may comprise a transmembrane region of CD8α.

The extracellular domain may be connected to the transmembrane region by a hinge region. The hinge region may comprise a hinge region of CD8α.

The CAR may comprise a signal peptide (SP), such as a CD8α signal peptide.

The CAR may be a BCMA CAR. A wide variety of antigen binding domain sequences can be used as the antigen binding domain of the CAR. The BCMA CAR may comprise from the N-terminus to the C-terminus: an anti-BCMA antibody, a CD8α hinge region, a CD8α transmembrane domain, a 4-1BB co-stimulatory signaling domain, and a CD3ζ intracellular signaling domain.

The BCMA CAR may comprise from the N-terminus to the C-terminus: a CD8α signal peptide, an anti-BCMA binder sequence, a CD8α hinge region, a CD8α transmembrane region, a 4-1BB co-stimulatory signaling domain, and a CD3 intracellular signaling domain. The BCMA CAR may comprise a first VHH antibody moiety that comprises a CDR1 comprising the amino acid sequence of SEQ ID NO: 29 or a variant thereof comprising up to about 3 amino acid substitutions, a CDR2 comprising the amino acid sequence of SEQ ID NO: 30 or a variant thereof comprising up to about 3 amino acid substitutions, and a CDR3 comprising the amino acid sequence of SEQ ID NO: 31 or a variant thereof comprising up to about 3 amino acid substitutions, and a second VHH antibody moiety that comprises a CDR1 comprising the amino acid sequence of SEQ ID NO: 32 or a variant thereof comprising up to about 3 amino acid substitutions, a CDR2 comprising the amino acid sequence of SEQ ID NO:33 or a variant thereof comprising up to about 3 amino acid substitutions, and a CDR3 comprising the amino acid sequence of SEQ ID NO: 34 or a variant thereof comprising up to about 3 amino acid substitutions. CDR sequences can be determined according to well-known numbering systems. In some embodiments, the CDRs are according to Kabat numbering.

The BCMA CAR may comprise a first VHH antibody moiety comprising the amino acid sequences of SEQ ID NO: 27 (i.e. BCMA 269A37948 VHH) or an amino acid sequence having at least 75%, 80%, 90%, 91%, 92%, 93%, 94%, 95%, 96%, 97%, 98%, or 99% identity to the amino acid sequence of SEQ ID NO: 27, and a second VHH antibody moiety comprising the amino acid sequences of SEQ ID NO: 28 (i.e. BCMA 269AS34822 VHH) or an amino acid sequence having at least 75%, 80%, 90%, 91%, 92%, 93%, 94%, 95%, 96%, 97%, 98%, or 99% identity to the amino acid sequence of SEQ ID NO: 28.

The BCMA CAR may comprise an amino acid sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 1. The BCMA CAR can have 1, 2, 3, 4, 5, 6, 7, 8, 9, 10 or more than 10 mutations (e.g., as compared to SEQ ID NO: 1). These mutations can be amino acid insertions, deletions, or substitutions. The insertions, deletions, and substitutions can be within the BCMA CAR, and/or at one or both terminal ends of the BCMA CAR. The CAR can specifically bind to BCMA-positive tumor cells (e.g., H929 cells).

The BCMA CAR may be co-expressed with the modified IL-15 or the related fusion proteins. The BCMA CAR may be linked to the modified IL-15 or the related fusion proteins via a P2A linker. The BCMA CAR may comprise an amino acid sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 2.

The disclosure may provide a vector encoding the BCMA CAR. The disclosure provides a vector encoding both the BCMA CAR and the modified IL-15 or the related fusion proteins. The disclosure provides a vector encoding both the BCMA CAR and the modified IL-15 or the related fusion proteins, linked by a P2A linker. The vector may be a lentiviral vector.

The engineered receptor may be a modified T-cell receptor. The engineered TCR may be specific for a tumor antigen. The tumor antigen may be selected from the group consisting of CD19, BCMA, NY-ESO-1, VEGFR2, MAGE-A3, VEGFR2, MAGE-A3, CD20, CD22, CD30, CD33, CD38, CEA, CS1, CD138, CD123/IL3Rα, c-Met, gp100, MUC1, IGF-I receptor, EpCAM, CEA, EGFR (such as EGFRvIII), GD2, HER2, IGF1R, mesothelin, PSMA, ROR1, WT1, Glypican 3 (GPC3), Guanylate cyclase 2C (GCC), DLL3, Claudin18.2, Claudin6, Glycolipid F77, PD-L1, PD-L2, and other tumor antigens with clinical significance, and combinations thereof. The tumor antigen may be derived from an intracellular protein of tumor cells. The tumor antigen may be expressed on the surface of tumor cells. Many TCRs specific for tumor antigens (including tumor-associated antigens) have been described, including, for example, NY-ESO-1 cancer-testis antigen, the p53 tumor suppressor antigens, TCRs for tumor antigens in melanoma (e.g., MARTI, gp 100), leukemia (e.g., WT1, minor histocompatibility antigens), and breast cancer (e.g., HER2, NY-BR1). Any of the TCRs known in the art can be used. The TCR may have an enhanced affinity to the tumor antigen. Exemplary TCRs and methods for introducing the TCRs to immune cells have been described, for example, in U.S. Pat. No. 5,830,755, and Kessels et al. Immunotherapy through TCR gene transfer. Nat. Immunol. 2, 957-961 (2001), which are incorporated herein by reference in the entirety.

The TCR receptor complex is an octomeric complex formed by variable TCR receptor α and β chains (or γ and δ chains on case of γδ T cells) with three dimeric signaling modules CD3δ/ε, CD3γ/ε and CD247 (T-cell surface glycoprotein CD3 zeta chain) ζ/ζ or ζ/η. Ionizable residues in the transmembrane region of each subunit form a polar network of interactions that hold the complex together. TCR complex has the function of activating signaling cascades in T cells.

The engineered receptor may be an engineered TCR comprising one or more T-cell receptor (TCR) fusion proteins (TFPs). Exemplary TFPs have been described, for example, in US20170166622A1, which is incorporated herein by reference in its entirety. The TFP may comprise an extracellular domain of a TCR subunit that comprises an extracellular domain or portion thereof of a protein selected from the group consisting of a TCR alpha chain, a TCR beta chain, a CD3 epsilon TCR subunit, a CD3 gamma TCR subunit, a CD3 delta TCR subunit, functional fragments thereof, and amino acid sequences thereof having at least one but not more than 20 modifications. The TFP may comprise a transmembrane region that comprises a transmembrane region of a protein selected from the group consisting of a TCR alpha chain, a TCR beta chain, a CD3 epsilon TCR subunit, a CD3 gamma TCR subunit, a CD3 delta TCR subunit, functional fragments thereof, and amino acid sequences thereof having at least one but not more than 20 modifications. The TFP may comprise a transmembrane region that comprises a transmembrane region of a protein selected from the group consisting of a TCR alpha chain, a TCR beta chain, a TCR zeta chain, a CD3 epsilon TCR subunit, a CD3 gamma TCR subunit, a CD3 delta TCR subunit, CD45, CD4, CD5, CD8, CD9, CD16, CD22, CD33, CD28, CD37, CD64, CD80, CD86, CD134, CD137, CD154, functional fragments thereof, and amino acid sequences thereof having at least one but not more than 20 modifications.

The engineered receptor may be a T-cell antigen coupler (TAC) receptor. Exemplary TAC receptors have been described, for example, in US20160368964A1, which is incorporated herein by reference. The TAC may comprise an antigen binding domain, a TCR-binding domain that specifically binds a protein associated with the TCR complex, and a T-cell receptor signaling domain. The antigen binding domain may be an antibody fragment, such as scFv or VHH, which specifically binds to a tumor antigen. The antigen binding domain may be a designed Ankyrin repeat (DARPin) polypeptide. The tumor antigen may be selected from the group consisting of CD19, BCMA, NY-ESO-1, VEGFR2, MAGE-A3, VEGFR2, MAGE-A3, CD20, CD22, CD30, CD33, CD38, CEA, CS1, CD138, CD123/IL3Rα, c-Met, gp100, MUC1, IGF-I receptor, EpCAM, CEA, EGFR (such as EGFRvIII), GD2, HER2, IGF1R, mesothelin, PSMA, ROR1, WT1, Glypican 3 (GPC3), Guanylate cyclase 2C (GCC), DLL3, Claudin18.2, Claudin6, Glycolipid F77, PD-L1, PD-L2, and other tumor antigens with clinical significance, and combinations thereof. The tumor antigen may be derived from an intracellular protein of tumor cells. The tumor antigen may be expressed on the surface of tumor cells. The protein associated with the TCR complex may be CD3, such as CD3E. The TCR-binding domain may be a single chain antibody, such as scFv, or a VHH. The TCR-binding domain may be derived from UCHT1. The TAC receptor may comprise a cytosolic domain and a transmembrane region. The T-cell receptor signaling domain may comprise a cytosolic domain derived from a TCR co-receptor. Exemplary TCR co-receptors include, but are not limited to, CD4, CD8, CD28, CD45, CD4, CD5, CD9, CD16, CD22, CD33, CD37, CD64, CD80, CD86, CD134, CD137 and CD 154. The TAC receptor may comprise a transmembrane region and a cytosolic domain derived from CD4. The TAC receptor may comprise a transmembrane region and a cytosolic domain derived from CD8 (such as CD8α).

T cell co-receptors are expressed as membrane protein on T cells. They can provide stabilization of the TCR: peptide: MEC complex and facilitate signal transduction. The two subtypes of T cell co-receptor, CD4 and CD8, display strong specificity for particular MEC classes. The CD4 co-receptor can only stabilize TCR: MEC II complexes while the CD8 co-receptor can only stabilize the TCR: MEC I complex. The differential expression of CD4 and CD8 on different T cell types results in distinct T cell functional subpopulations. CD8+ T cells are cytotoxic T cells.

The engineered receptor (such as CAR, TCR, or TAC) may target one or more tumor antigens. Tumor antigens are proteins that are produced by tumor cells that can elicit an immune response, particularly T-cell mediated immune responses. The selection of the targeted antigen will depend on the particular type of cancer to be treated. Exemplary tumor antigens include, for example, a glioma-associated antigen, carcinoembryonic antigen (CEA), β-human chorionic gonadotropin, alphafetoprotein (AFP), lectin-reactive AFP, thyroglobulin, RAGE-1, MN-CAIX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-la, p53, prostein, PSMA, HER2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

The tumor antigen may comprise one or more antigenic cancer epitopes associated with a malignant tumor. Malignant tumors express a number of proteins that can serve as target antigens for an immune attack. These molecules include but are not limited to tissue-specific antigens such as MART-1, tyrosinase and gp 100 in melanoma and prostatic acid phosphatase (PAP) and prostate-specific antigen (PSA) in prostate cancer. Other target molecules belong to the group of transformation-related molecules such as the oncogene HER2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B cell differentiation antigens such as CD 19, CD20 and CD37 are other candidates for target antigens in B-cell lymphoma.

The tumor antigen may be a tumor-specific antigen (TSA) or a tumor-associated antigen (TAA). A TSA is unique to tumor cells and does not occur on other cells in the body. A TAA associated antigen is not unique to a tumor cell, and instead is also expressed on a normal cell under conditions that fail to induce a state of immunologic tolerance to the antigen. The expression of the antigen on the tumor can occur under conditions that enable the immune system to respond to the antigen. TAAs can be antigens that are expressed on normal cells during fetal development, when the immune system is immature, and unable to respond or they can be antigens that are normally present at extremely low levels on normal cells, but which are expressed at much higher levels on tumor cells.

Non-limiting examples of TSA or TAA antigens include the following: Differentiation antigens such as MART-1/MelanA (MART-I), gp 100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23HI, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS 1, SDCCAG16, TA-90\Mac-2 binding protein\cyclophilin C-associated protein, TAAL6, TAG72, TLP, and TPS.

The CAR may comprise a primary intracellular signaling domain of an immune effector cell, and/or a co-stimulatory signaling domain. The primary intracellular signaling domain may be derived from CD32, and wherein the co-stimulatory signaling domain is derived from a co-stimulatory molecule selected from the group consisting of 4-1BB, CD27, CD28, CD137, OX40, CD30, CD40, CD3, LFA-1, ICOS, CD2, CD7, LIGHT, NKG2C, B7-H3, ligands of CD83 and combinations thereof. The CAR may comprise a transmembrane domain derived from a molecule selected from the group consisting of CD8α, CD4, CD28, CD137, CD80, CD86, CD152 and PD1. The CAR may further comprise a hinge domain located between the C-terminus of the extracellular antigen binding domain and the N-terminus of the transmembrane domain. The CAR may further comprise a signal peptide located at the N-terminus of the polypeptide.

### Nucleic Acids (Polynucleotides) Encoding the Modified IL-15 or the Related Fusion Proteins and Related Polypeptides

The present disclosure provides nucleic acids (e.g., expression vectors) encoding the modified IL-15 or the related fusion proteins. The present disclosure also provides (i)nucleic acids (e.g., expression vectors) encoding the modified IL-15 or the related fusion proteins, and (ii)nucleic acids (e.g., expression vectors) encoding an engineered receptor (e.g., CAR or TCR). The nucleic acids of the present disclosure can comprise a nucleic acid sequence encoding any one of the modified IL-15 or the related fusion proteins, CARs, and/or TCRs disclosed herein. The nucleic acid sequence may encode for both a CAR and a modified IL-15 or the related fusion proteins (CAR armored with modified IL-15 or the related fusion proteins). The nucleic acid sequence may encode for a CAR and a modified IL-15 or the related fusion proteins, linked to each other by a P2A linker.

A polynucleotide of the present disclosure may comprise a first polynucleotide sequence and a second polynucleotide sequence. The first and second polynucleotide sequence can be separated by a linker. A linker for use in the present disclosure allows for multiple proteins to be encoded by the same nucleic acid sequence (e.g., a multicistronic or bicistronic sequence), which are translated as a polyprotein that is dissociated into separate protein components. The polynucleotide may comprise from 5′ to 3′ the first polynucleotide sequence, the linker, and the second polynucleotide sequence. The polynucleotide may comprise from 5′ to 3′ the second polynucleotide sequence, the linker, and the first polynucleotide sequence. The first polynucleotide sequence may encode an engineered receptor (e.g., CAR) described herein and the second polynucleotide sequence encodes a modified IL-15 or the related fusion proteins described herein.

The linker may comprise a nucleic acid sequence that encodes for an internal ribosome entry site (IRES). As used herein, “an internal ribosome entry site” or “IRES” refers to an element that promotes direct internal ribosome entry to the initiation codon, such as ATG, of a protein coding region, thereby leading to cap-independent translation of the gene. Various internal ribosome entry sites are known to those of skill in the art, including, without limitation, IRES obtainable from viral or cellular mRNA sources, e.g., immunogloublin heavy-chain binding protein (BiP); vascular endothelial growth factor (VEGF); fibroblast growth factor 2; insulin-like growth factor; translational initiation factor eIF4G; yeast transcription factors TFIID and HAP4; and IRES obtainable from, e.g., cardiovirus, rhinovirus, aphthovirus, HCV, Friend murine leukemia virus (FrMLV), and Moloney murine leukemia virus (MoMLV). Those of skill in the art would be able to select the appropriate IRES.

The linker may comprise a nucleic acid sequence that encodes for a self-cleaving peptide. As used herein, a “self-cleaving peptide” or “2A peptide” refers to an oligopeptide that allow multiple proteins to be encoded as polyproteins, which dissociate into component proteins upon translation. Use of the term “self-cleaving” is not intended to imply a proteolytic cleavage reaction. Various self-cleaving or 2A peptides are known to those of skill in the art, including, without limitation, those found in members of the Picornaviridae virus family, e.g., foot-and-mouth disease virus (FMDV), equine rhinitis A virus (ERAVO, Thosea asigna virus (TaV), and porcine tescho virus-1 (PTV-1); and carioviruses such as Theilovirus and encephalomyocarditis viruses.2A peptides derived from FMDV, ERAV, PTV-1, and TaV are referred to herein as “F2A,” “E2A,” “P2A,” and “T2A,” respectively. Those of skill in the art would be able to select the appropriate self-cleaving peptide. The P2A linker may have a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 2.

The linker can comprise a spacer sequence. Various spacer sequences are known in the art, including, without limitation, glycine serine (GS) spacers (also known as GS linkers) such as (GS)n, (SG)n, (GSGGS)n and (GGGS)n, where n represents an integer of at least 1. Exemplary spacer sequences can comprise amino acid sequences including, without limitation, GGSG, GGSGG, GSGSG, GSGGG, GGGSG, GSSSG, and the like. Those of skill in the art would be able to select the appropriate spacer sequence.

The polynucleotide of the present disclosure may comprise a restriction enzyme site sequence.

The polynucleotide of the present disclosure can be operably linked to a transcriptional control element, e.g., a promoter, and enhancer, etc. Suitable promoter and enhancer elements are known to those of skill in the art.

Suitable promoters include the immediate early cytomegalovirus (CMV) promoter sequence. This promoter sequence is a strong constitutive promoter sequence capable of driving high levels of expression of any polynucleotide sequence operatively linked thereto. Other constitutive promoter sequences can also be used, including, but not limited to a simian virus 40 (SV40) early promoter, a mouse mammary tumor virus (MMTV) or human immunodeficiency virus (HIV) long terminal repeat (LTR) promoter, a MoMuLV promoter, an avian leukemia virus promoter, an Epstein-Barr virus immediate early promoter, a Rous sarcoma virus promoter, the EF-1 alpha promoter, as well as human gene promoters such as, but not limited to, an actin promoter, a myosin promoter, a hemoglobin promoter, and a creatine kinase promoter. Further, the disclosure should not be limited to the use of constitutive promoters. Inducible promoters are also contemplated as part of the disclosure. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

The polynucleotide of the present disclosure can be provided for the production of (i) a modified IL-15 or the related fusion proteins described herein, and/or (ii) a CAR described herein (e.g., in a mammalian cell). The polynucleotide of the present disclosure may provide for amplification of the polynucleotide.

An expression vector (e.g., a lentiviral vector) can be used to introduce the modified IL-15 or the related fusion proteins, CAR and/or TCR into an immune cell or precursor thereof (e.g., a T cell). Accordingly, an expression vector (e.g., a lentiviral vector) of the present disclosure can comprise a polynucleotide encoding for the modified IL-15 or the related fusion proteins, CAR and/or TCR. The expression vector (e.g., lentiviral vector) will comprise additional elements that will aid in the functional expression of the modified IL-15 or the related fusion proteins, CAR and/or TCR encoded therein. An expression vector comprising a polynucleotide may further comprise a mammalian promoter. The vector may further comprise an elongation-factor-1-alpha promoter (EF-1α promoter). Use of an EF-1α promoter can increase the efficiency in expression of downstream transgenes (e.g., a CAR- or TCR-encoding polynucleotide). Physiologic promoters (e.g., an EF-1α promoter) can be less likely to induce integration mediated genotoxicity, and can abrogate the ability of the retroviral vector to transform stem cells. Other physiological promoters suitable for use in a vector (e.g., lentiviral vector) are known to those of skill in the art and can be incorporated into a vector of the present disclosure. The vector (e.g., lentiviral vector) may further comprise a non-requisite cis acting sequence that can improve titers and gene expression.

The polynucleotide may encode a naked CAR. The polynucleotide may comprise from the 5′ end to the 3′ end, an antigen binding domain, a CD8α hinge region, a CD8α transmembrane region, a 4-1BB co-stimulatory signaling domain, a CD3ζ cytoplasmic domain. The amino acid sequence of the CAR can be at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to in SEQ ID NO: 1.

The polynucleotide may encode a CAR and a modified IL-15 or the related fusion proteins. The polynucleotide may comprise from the 5′ end to the 3′ end, the coding sequences of an antigen binding domain, a CD8α hinge region, a CD8α transmembrane region, a 4-1BB co-stimulatory signaling domain, a CD33 cytoplasmic domain, a 2A cleavable linker, a modified IL-15 or the related fusion proteins. The polynucleotide may encode a BCMA CAR and a modified IL-15 or the related fusion proteins. The amino acid sequence of the modified IL-15 or the related fusion proteins may be at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOs: 17-26.

The present disclosure may provide (i) polypeptides including the modified IL-15 or the related fusion proteins, and (ii) polypeptides including the engineered receptor (e.g., CAR or TCR). The polypeptides of the present disclosure can comprise any one of the modified IL-15 or the related fusion proteins, CARs, and/or TCRs disclosed herein. The polypeptide may include both a CAR and a modified IL-15 or the related fusion proteins (CAR armored with modified IL-15 or the related fusion proteins).

A polypeptide of the present disclosure may comprise a first polypeptide sequence and a second polypeptide sequence. The first and second polypeptide sequences can be separated by a linker. A linker for use in the present disclosure allows for multiple proteins to be encoded by the same nucleic acid sequence (e.g., a multicistronic or bicistronic sequence), which are translated as a polyprotein that is dissociated into separate protein components. The polypeptide may comprise from N-terminus to C-terminus: the first polypeptide sequence, the linker, and the second polypeptide sequence. The polypeptide may comprise from N-terminus to C-terminus: the second polypeptide sequence, the linker, and the first polypeptide sequence. The first polypeptide sequence may contain the polypeptide sequence of an engineered receptor (e.g., CAR) described herein and the second polypeptide sequence contains the polypeptide sequence of a modified IL-15 or the related fusion proteins described herein.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein. The disclosure relates to nucleotide sequences encoding any peptides that are described herein, or any amino acid sequences that are encoded by any nucleotide sequences as described herein. The nucleic acid sequence may be less than 10, 20, 30, 40, 50, 60, 70, 80, 90, 100, 110, 120, 130, 150, 200, 250, 300, 350, 400, 500, 600, 800, 1000, 1200, 1400, 1600, 1800, 2000, 2500, 3000, 3500, 4000, or 5000 nucleotides. The amino acid sequence may be less than 5, 6, 7, 8, 9, 10, 20, 30, 40, 50, 60, 70, 80, 90, 100, 110, 120, 130, 140, 150, 160, 170, 180, 190, 200, 220, 240, 260, 280, 300, 350, 400, 450, 500, 550, 600, 700, 800, 900, 1000, 1100, 1200, 1300, or 1400 amino acid residues.

The amino acid sequence may (i) comprise an amino acid sequence; or (ii) consist of an amino acid sequence, wherein the amino acid sequence is any one of the sequences as described herein.

The nucleic acid sequence may (i) comprise a nucleic acid sequence; or (ii) consist of a nucleic acid sequence, wherein the nucleic acid sequence is any one of the sequences as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and may be at least 90%, 95%, or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

Introduction of Polynucleotides into Host Cells

The polynucleotides (e.g., vectors) described herein can be introduced as one or more polynucleotides or constructs, optionally comprising a marker that will allow for selection of host cells that contain the construct(s). The genes and regulatory regions can be isolated, as appropriate, ligated, cloned in an appropriate cloning host, analyzed by restriction or sequencing. Particularly, using PCR, individual fragments including all or portions of a functional unit can be isolated, where one or more mutations can be introduced using “primer repair”, ligation, in vitro mutagensis, etc. as appropriate. The polynucleotides obtained and demonstrated to have the appropriate sequences can then be introduced into the host cell by any convenient means. The polynucleotides can be integrated and packaged into non-replicating, defective viral genomes like lentivirus, Adenovirus, Adeno-associated virus (AAV), or Herpes simplex virus (HSV) or others, including retroviral vectors, for infection or transduction into cells. The polynucleotides can include viral sequences for transfection, if desired. Alternatively, the polynucleotides can be introduced by fusion, electroporation, biolistics, transfection, lipofection, or the like. The host cells can be grown and expanded in culture before introduction of the construct(s), followed by the appropriate treatment for introduction of the construct(s) and integration of the construct(s). The cells are then expanded and screened by virtue of a marker present in the construct. Various markers that can be used successfully include hprt, neomycin resistance, thymidine kinase, hygromycin resistance, etc.

The nucleic acids encoding modified IL-15 or the related fusion proteins and/or engineered receptor can be introduced into the modified cells as an RNA for transient expression. RNA can be delivered to the immune cells of the disclosure by various means including microinjection, electroporation, and lipid-mediated transfection, for example. Introduction of constructs into the cell's genome can occur via transposons. An example of a synthetic transposon for use is the Sleeping Beauty transposon that comprises an expression cassette including the appropriate gene of active fragment thereof. The construct can be integrated at a particular locus in the genome of the host cell. An endogenous gene can be replaced with the gene encoded for by the construct using homologous recombination.

A construct encoding both a modified IL-15 or the related fusion proteins and a CAR can be introduced into the host cell using a lentiviral delivery system. A construct encoding both a modified IL-15 or the related fusion proteins and a CAR can be introduced into the host cell using a retroviral delivery system.

The host cells may be human cells. The host cells may be human T cells. The human T cells may be purified from commercialized PBMCs using Miltenyi Pan T cell isolation kit (Cat #130-096-535), following manufacturer's protocols. The host cells may be αβT cells. The host cells may be γδT cells. The host cells may be Vδ1 T cells. The host cells may be NK cells.

### Modified Cells

The present disclosure provides modified cells comprising the modified IL-15 or the related fusion proteins described herein. The modified cells comprising the modified IL-15 or the related fusion proteins described herein may further comprise an engineered receptor (e.g., CAR). The modified cells comprising the modified IL-15 or the related fusion proteins described herein may further comprise a CAR (CAR armored with modified IL-15 or the related fusion proteins). The cell may be an immune cell. The cell may be selected from a group consisting of T cell, αβT cell, γδT cell, NK cell, peripheral blood mononuclear cell (PBMC), hematopoietic stem cell, pluripotent stem cell, an embryonic stem cell, and a combination thereof.

The engineered receptor (e.g., CAR) can redirect the specificity of the modified cells through the expression of a chimeric antigen receptor (CAR) or TCR on these cells. CAR expression can be induced through electroporation of modified cells for the insertion of genetic material, or by infecting these cells with viral vectors, such as lentiviruses or retroviruses containing the desired genetic material. Such genetic editing can improve the potency of the modified cells by improving homing, cytokine production, recycle killing, and/or improved engraftment.

The modified cells comprising the modified IL-15 or the related fusion proteins described herein may express more than one engineered receptor, such as any combination of CAR, TCR, or TAC receptors.

The modified cell comprising the modified IL-15 or the related fusion proteins described herein can be used to treat cancer.

Comparing to a cell without the modified IL-15 or the related fusion proteins described herein, the modified cell comprising the modified IL-15 or the related fusion proteins described herein may have a higher cytotoxicity against tumor cells. Comparing to a cell without the modified IL-15 or the related fusion proteins described herein, the modified cell comprising the modified IL-15 or the related fusion proteins described herein may have a higher persistence and/or proliferation in the tumor microenvironment.

The present disclosure provides modified cells comprising (i) a modified IL-15 or the related fusion proteins described herein and (ii) an engineered receptor (e.g., CAR) described herein. The modified cell may be an immune cell. The modified cell may comprise one or more polynucleotides encoding (i) a modified IL-15 or the related fusion proteins described herein and (ii) an engineered receptor (e.g., CAR) described herein. Accordingly, such modified cells possess the specificity directed by the engineered receptor (e.g., CAR) that is expressed therein. For example, a modified cell of the present disclosure comprising a CAR(s) possesses specificity for one or more antigen(s) on a target cell (e.g., one or more tumor antigen(s) on a cancer cell).

The modified cells may be modified immune cells. The modified cells may be T cells. The modified cells may be NK cells. The modified cells may be αβ T cells. The modified cells may be γδ T cells. The modified cells may be Vδ1 T cells.

The modified cells may be an autologous cells, syngeneic cells, allogeneic cells, or xenogeneic cells with respect to the individual receiving them. The modified cells can be modified by changing the major histocompatibility complex (MHC) profile, by inactivating β2-microglobulin to prevent the formation of functional Class I MHC molecules, or by inactivating Class II MHC molecules.

The modified cells described herein may include eukaryotic cells, e.g., mammalian cells. The modified cells may be human cells. The modified cells may be equine, bovine, murine, ovine, canine, or feline cells.

The modified cells may be autologous cells obtained from the human subject receiving them. The modified cells may be autologous T cells obtained from the human subject receiving them.

The present disclosure provides a modified cell that expresses a modified IL-15 or the related fusion proteins. The modified IL-15 or the related fusion proteins may contain a calreticulin signal peptide sequence. The modified IL-15 or the related fusion proteins may contain an ER retention sequence. The modified IL-15 or the related fusion proteins may contain a myristoylation sequence. The modified IL-15 or the related fusion proteins may be trapped in the ER. The modified IL-15 or the related fusion proteins may be trapped inside the cell. The modified IL-15 or the related fusion proteins may reduce the amount of secreted IL-15 by trapping it inside cells. The modified IL-15 or the related fusion proteins may activate IL-15 signaling.

Comparing to unmodified cells that do not comprise the modified IL-15 or the related fusion proteins, the modified cells may have an enhanced proliferation. Comparing to unmodified cells that do not comprise the modified IL-15 or the related fusion proteins, the modified cells may have an enhanced cytotoxicity.

The engineered receptor may be a CAR. The CAR may comprise a polypeptide comprising from N-terminus to C-terminus: an antigen binding domain, a CD8α hinge region, a CD8α transmembrane region, a 4-1BB co-stimulatory signaling domain, and a CD3ζ cytoplasmic domain. The CAR may be a BCMA CAR. The CAR may comprise a polypeptide comprising an amino acid sequence having at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to in SEQ ID NO: 1.

The polypeptide may comprise a CAR and a modified IL-15 or the related fusion proteins. The polypeptide may comprise from N-terminus to C-terminus: an antigen binding domain, a CD8α hinge region, a CD8α transmembrane region, a 4-1BB co-stimulatory signaling domain, a CD3ζ cytoplasmic domain, a 2A cleavable linker, a modified IL-15 or the related fusion proteins. The polypeptide may comprise a BCMA CAR and/or a modified IL-15 or the related fusion proteins. The amino acid sequence of the modified IL-15 or the related fusion proteins may be at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOs: 17-26.

The modified cells may comprise both a modified IL-15 or the related fusion proteins and a CAR (CAR armored with modified IL-15 or the related fusion proteins). The modified cells may be modified CAR-T cells (modified IL-15 or the related fusion proteins armored CAR-T cells). The expression of CAR and modified IL-15 or the related fusion proteins by the modified cells can be determined by flow cytometry (FACS). The modified cells may have a CAR positive rate of more than 5%, more than 10%, more than 15%, more than 20%, more than 25%, more than 30%, more than 35%, more than 40%, more than 45%, more than 50%, more than 55%, more than 60%, more than 65%, more than 70%, more than 75%, more than 80%, or more than 90%. The modified cells may have a CAR positive rate of less than 5%, less than 10%, less than 15%, less than 20%, less than 25%, less than 30%, less than 35%, less than 40%, less than 45%, less than 50%, less than 55%, less than 60%, less than 65%, less than 70%, less than 75%, less than 80%, or less than 90%. The modified cells may have a CAR positive rate of 10%-80%, 10%-70%, 15%-70%, 20%-70%, 20%-65%, 25%-65%, 30%-80%, 40%-80%, 50%-80%, or 60%-80%.

The modified cells may have an IL-15 positive rate of more than 5%, more than 10%, more than 15%, more than 20%, more than 25%, more than 30%, more than 35%, more than 40%, more than 45%, more than 50%, more than 55%, more than 60%, more than 65%, more than 70%, more than 75%, more than 80%, or more than 90%. The modified cells may have an IL-15 positive rate of less than 5%, less than 10%, less than 15%, less than 20%, less than 25%, less than 30%, less than 35%, less than 40%, less than 45%, less than 50%, less than 55%, less than 60%, less than 65%, less than 70%, less than 75%, less than 80%, or less than 90%. The modified cells may have an IL-15 positive rate of 10%-80%, 10%-70%, 15%-70%, 20%-70%, 20%-65%, 20%-60%, 20%-55%, 25%-65%, 30%-70%, 30%-75%, 30%-65%, 20%-40%, 20%-50%, 20%-60%, 20%-70%, 30%-70%, or 40%-70%.

The modified cells may have an IL-15Rα positive rate of more than 5%, more than 10%, more than 15%, more than 20%, more than 25%, more than 30%, more than 35%, more than 40%, more than 45%, more than 50%, more than 55%, more than 60%, more than 65%, more than 70%, more than 75%, more than 80%, or more than 90%. The modified cells may have an IL-15Rα positive rate of less than 5%, less than 10%, less than 15%, less than 20%, less than 25%, less than 30%, less than 35%, less than 40%, less than 45%, less than 50%, less than 55%, less than 60%, less than 65%, less than 70%, less than 75%, less than 80%, or less than 90%. The modified cells may have an IL-15Rα positive rate of 10%-80%, 10%-70%, 15%-70%, 20%-70%, 20%-65%, 20%-60%, 20%-55%, 25%-65%, 30%-70%, 30%-75%, 30%-65%, 20%-40%, 20%-50%, 20%-60%, 20%-70%, 30%-70%, or 40%-70%.

In one aspect, the disclosure provides methods to increase cell viability, the methods comprising expressing the modified IL-15 or the related fusion protein in the cell. The viability of the modified cells can be assessed by an in vitro cell culture assay. The viability of the modified cells can be assessed by an in vitro cell culture assay in cytokine-free medium. The modified cells may have a viability of more than 5%, more than 10%, more than 15%, more than 20%, more than 25%, more than 30%, more than 35%, more than 40%, more than 45%, more than 50%, more than 55%, more than 60%, more than 65%, more than 70%, more than 75%, more than 80%, or more than 90%, after 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, or 49 days. The modified cells may have a viability of less than 5%, less than 10%, less than 15%, less than 20%, less than 25%, less than 30%, less than 35%, less than 40%, less than 45%, less than 50%, less than 55%, less than 60%, less than 65%, less than 70%, less than 75%, less than 80%, or less than 90%, after 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, or 49 days. Comparing to unmodified cells that do not comprise the modified IL-15 or the related fusion proteins, the modified cells comprising the modified IL-15 or the related fusion proteins may have a similar viability, after 1 round, 2 rounds, 3 rounds, 4 rounds, or 5 rounds of stimulation in a re-challenge assay.

In one aspect, the disclosure provides methods to increase cell proliferation, the methods comprising expressing the modified IL-15 or the related fusion protein in the cell. The proliferation of the modified cells can be assessed by an in vitro cell culture assay. The proliferation of the modified cells can be assessed by an in vitro cell culture assay in cytokine-free medium. The proliferation of the modified cells can be assessed by cell counting. The proliferation of the modified cells can be assessed by a dye dilution method. The modified cells may be amplified by more than 1 fold, more than 2 fold, more than 3 fold, more than 4 fold, more than 5 fold, more than 6 fold, more than 7 fold, more than 8 fold, more than 9 fold, more than 10 fold, more than 15 fold, more than 20 fold, more than 25 fold, more than 30 fold, more than 35 fold, more than 40 fold, more than 45 fold, more than 50 fold, more than 60 fold, more than 70 fold, more than 80 fold, more than 90 fold, more than 100 fold, more than 110 fold, more than 120 fold, after 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, or 49 days. The modified cells may be amplified by less than 1 fold, less than 2 fold, less than 3 fold, less than 4 fold, less than 5 fold, less than 6 fold, less than 7 fold, less than 8 fold, less than 9 fold, less than 10 fold, less than 15 fold, less than 20 fold, less than 25 fold, less than 30 fold, less than 35 fold, less than 40 fold, less than 45 fold, less than 50 fold, less than 60 fold, less than 70 fold, less than 80 fold, less than 90 fold, less than 100 fold, less than 110 fold, less than 120 fold, after 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, or 49 days. The initial number of modified cells may be below 1 million. The number of modified cells may be above 1 million, above 2 million, above 3 million, above 4 million, above 5 million, above 6 million, above 7 million, above 8 million, above 9 million, above 10 million, above 11 million, above 12 million, above 13 million, above 14 million, above 15 million, above 16 million, above 17 million, above 18 million, above 19 million, above 20 million, above 21 million, above 22 million, above 23 million, above 24 million, above 25 million, after 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, or 49 days. The number of modified cells may be below 1 million, below 2 million, below 3 million, below 4 million, below 5 million, below 6 million, below 7 million, below 8 million, below 9 million, below 10 million, below 11 million, below 12 million, below 13 million, below 14 million, below 15 million, below 16 million, below 17 million, below 18 million, below 19 million, below 20 million, below 21 million, below 22 million, below 23 million, below 24 million, below 25 million, after 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, or 49 days.

The modified cells may have activated STAT signaling (e.g., phosphorylated STAT3, phosphorylated STAT5, or phosphorylated STAT6). The modified cells may have phosphorylated STAT5.

In one aspect, the disclosure provides methods to increase a cell's STAT signaling (e.g., increase the amount of phosphorylated STAT5), the methods comprising expressing the modified IL-15 or the related fusion protein in the cell. Comparing to unmodified cells that do not comprise the modified IL-15 or the related fusion proteins, the amount of phosphorylated STAT5 in the modified cells may be increased by more than 5%, more than 10%, more than 15%, more than 20%, more than 30%, more than 40%, more than 50%, more than 60%, more than 70%, more than 80%, more than 90%, more than 100%, more than 150%, more than 200%, more than 250%, more than 300%, more than 400%, more than 500%, more than 600%, more than 700%, more than 800%, more than 900%, or more than 10,00%.

In one aspect, the disclosure provides methods to increase a cell's cytotoxicity against a tumor cell, the methods comprising expressing the modified IL-15 or the related fusion protein in the cell. The modified cells can kill tumor cells. The cytotoxicity of the modified cells against tumor cells (e.g., H929 cells) can be determined by an in vitro cytotoxicity assay. The effector cell:target cell (E:T) ratio may be 0.5:1, 1:1, 2:1, 2.5:1, 5:1, 10:1, or 1:4. The E:T ratio may be 1:4. The tumor cells may be labeled with cell trace violet. Fresh tumor cells (e.g., H929 cells) were added every 24 hours to examine the cytotoxicity of the modified cells after multiple rounds of challenge. The modified cells may have a cytotoxicity of more than 5%, more than 10%, more than 15%, more than 20%, more than 30%, more than 40%, more than 50%, more than 60%, more than 70%, more than 80%, more than 90%, or more than 95%, after 1 round, 2 rounds, 3 rounds, 4 rounds, 5 rounds, 6 rounds, 7 rounds, or 8 rounds of challenge in a re-challenge assay. The modified cells may have a cytotoxicity of less than 5%, less than 10%, less than 15%, less than 20%, less than 30%, less than 40%, less than 50%, less than 60%, less than 70%, less than 80%, less than 90%, or less than 95%, after 1 round, 2 rounds, 3 rounds, 4 rounds, 5 rounds, 6 rounds, 7 rounds, or 8 rounds of challenge in a re-challenge assay. The modified cells may have a cytotoxicity of 10-100%, 10%-50%, 20-100%, 20-60%, 20-40%, 30-70%, 40-80%, 50-90%, 60-100%, 70-100%, 80-100%, or 90-100%, after 1 round, 2 rounds, 3 rounds, 4 rounds, 5 rounds, 6 rounds, 7 rounds, or 8 rounds of challenge in a re-challenge assay.

The modified IL-15 or the related fusion proteins may also be co-expressed with an IL-15 receptor alpha subunit. Similarly, various other proteins (besides a CD16 and/or CAR may be co-expressed with the modified IL-15 or the related fusion proteins, and suitable co-expressed proteins include various immune modulatory compounds, and especially compounds that interfere with checkpoint inhibition (e.g., scFv against PD 1, PD-L1, CTLA4, etc.), immune stimulation (e.g., IFN-g, IL-12, IL-21, etc.) and/or compounds that bind/inhibit cytokines involved with immune suppression (e.g., TGF-b, IL-8, etc.).

The modified cells will express the modified IL-15 or the related fusion proteins in an amount sufficient to (a) render the so transfected cells independent from exogenous cytokines, and (b) allow for stimulation/activation of other immune competent cells that are in proximity to the transfected cells (typically within the TME).

The secreted or otherwise extracellular (or extracellularly presented) IL-15 or IL-15 variant will account for more than 5%, more than 10%, more than 15%, more than 20%, more than 30%, more than 40%, more than 50%, more than 60%, more than 70%, more than 80%, more than 90%, or more than 95% of the total IL-15 or IL-15 variant produced in the modified cells. The secreted or otherwise extracellular (or extracellularly presented) IL-15 or IL-15 variant will account for less than 5%, less than 10%, less than 15%, less than 20%, less than 30%, less than 40%, less than 50%, less than 60%, less than 70%, less than 80%, less than 90%, or less than 95% of the total IL-15 or IL-15 variant produced in the modified cells.

The intracellularly retained IL-15 or IL-15 variant will account for more than 5%, more than 10%, more than 15%, more than 20%, more than 30%, more than 40%, more than 50%, more than 60%, more than 70%, more than 80%, more than 90%, or more than 95% of the total IL-15 or IL-15 variant produced in the cell. Intracellularly retained IL-15 or IL-15 variant will account for less than 5%, less than 10%, less than 15%, less than 20%, less than 30%, less than 40%, less than 50%, less than 60%, less than 70%, less than 80%, less than 90%, or less than 95% of the total IL-15 or IL-15 variant produced in the modified cells.

The modified cells will produce IL-15 or IL-15 variant in sufficient quantities to support autonomous growth and stimulate immune competent cells in the TME as well as to stimulate establishment and maintenance of CD8+ T cell memory, but that such quantities are insufficient to trigger a systemic adverse event in a subject receiving such cells.

The amount of secreted IL-15 can be measured by detecting the amount of IL-15 in the cell culture medium using an IL-15 ELISA assay. The amount of intracellular IL-15 can be measured by detecting the amount of IL-15 in the cell lysate using an IL-15 ELISA assay. 4 million modified cells were analyzed for the amount of secreted IL-15 and the amount of intracellular IL-15. The amount of secreted IL-15 may be more than 10 pg, more than 20 pg, more than 30 pg, more than 40 pg, more than 50 pg, more than 60 pg, more than 70 pg, more than 80 pg, more than 90 pg, more than 100 pg, more than 200 pg, more than 300 pg, more than 400 pg, more than 500 pg, more than 600 pg, more than 700 pg, more than 800 pg, more than 900 pg, more than 1000 pg, more than 1200 pg, more than 1400 pg, more than 1600 pg, more than 1800 pg, more than 2000 pg, more than 2200 pg, or more than 2400 pg. The amount of secreted IL-15 may be less than 10 pg, less than 20 pg, less than 30 pg, less than 40 pg, less than 50 pg, less than 60 pg, less than 70 pg, less than 80 pg, less than 90 pg, less than 100 pg, less than 200 pg, less than 300 pg, less than 400 pg, less than 500 pg, less than 600 pg, less than 700 pg, less than 800 pg, less than 900 pg, less than 1000 pg, less than 1200 pg, less than 1400 pg, less than 1600 pg, less than 1800 pg, less than 2000 pg, less than 2200 pg, or less than 2400 pg.

The amount of intracellular IL-15 may be more than 10 pg, more than 20 pg, more than 30 pg, more than 40 pg, more than 50 pg, more than 60 pg, more than 70 pg, more than 80 pg, more than 90 pg, more than 100 pg, more than 200 pg, more than 300 pg, more than 400 pg, more than 500 pg, more than 600 pg, more than 700 pg, more than 800 pg, more than 900 pg, more than 1000 pg, more than 1200 pg, more than 1400 pg, more than 1600 pg, more than 1800 pg, more than 2000 pg, more than 2200 pg, or more than 2400 pg. The amount of intracellular IL-15 may be less than 10 pg, less than 20 pg, less than 30 pg, less than 40 pg, less than 50 pg, less than 60 pg, less than 70 pg, less than 80 pg, less than 90 pg, less than 100 pg, less than 200 pg, less than 300 pg, less than 400 pg, less than 500 pg, less than 600 pg, less than 700 pg, less than 800 pg, less than 900 pg, less than 1000 pg, less than 1200 pg, less than 1400 pg, less than 1600 pg, less than 1800 pg, less than 2000 pg, less than 2200 pg, or less than 2400 pg.

The ratio between the secreted IL-15 and the intracellular IL-15 may be more than 0.1, more than 0.2, more than 0.3, more than 0.4, more than 0.5, more than 0.6, more than 0.7, more than 0.8, more than 0.9, more than 1, more than 2, more than 3, more than 4, more than 5, more than 6, more than 7, more than 8, more than 9, or more than 10. The ratio between the secreted IL-15 and the intracellular IL-15 may be less than 0.1, less than 0.2, less than 0.3, less than 0.4, less than 0.5, less than 0.6, less than 0.7, less than 0.8, less than 0.9, less than 1, less than 2, less than 3, less than 4, less than 5, less than 6, less than 7, less than 8, less than 9, or less than 10. The ratio between the secreted IL-15 and the intracellular IL-15 may be 0.1-0.2, 0.1-0.3, 0.1-0.4, 0.1-0.5, 0.2-0.5, 0.3-0.5, or 0.4-0.5.

The modified IL-15 or the related fusion proteins can stimulate or enhance effector functions and/or proliferation of NK-cells (e.g., autologous NK cells in a TME), various T cells, etc., as well as enhance or trigger Jak/STAT signaling in cells in the TME. Due to the fraction of intracellularly retained IL-15 or IL-15 variant, the modified cells may also be able to proliferate in the complete absence of exogenous IL-2 and/or IL-15. The modified cells may also have an increased sensitivity to IL-12 signaling as compared to unmodified cells, which may reduce IL-4 mediated suppression of IFN-g, which in turn may reduce suppression of Thl T cells in the TME.

### Methods of Treatment

The modified IL-15 or the related fusion proteins described herein, the polynucleotides described herein and the modified cells described herein can be used in a variety of experimental, therapeutic and commercial applications.

In one aspect, the disclosure provides a method of modulating an immune response comprising administering an effective amount of modified cells described herein to a subject in need thereof.

The term “effective amount” as used herein means an amount effective, at dosages and for periods of time necessary to achieve the desired results.

In another aspect, the present disclosure provides a method of treating an infection comprising administering an effective amount of modified cells described herein to a subject in need thereof.

Examples of infections that can be treated include, but are not limited to, bacterial infections such as those caused by Mycobacteria (e.g., tuberculosis), viral infections such as those caused by herpes simplex virus (HSV), human immunodeficiency virus (HIV) or the hepatitis viruses, and parasitic infections such as those caused by Plasmodium (e.g., malaria).

In another aspect, the present disclosure provides a method for treating cancer comprising administering an effective amount of modified cells described herein to a subject in need thereof.

Examples of cancer that can be treated include, but are not limited to, leukemias including chronic lymphocytic leukemia, chronic myelogenous leukemia, acute myelogenous leukemia, acute lymphoblastic leukemia, and T cell and B cell leukemias, lymphomas (Hodgkin's and non-Hodgkins), lymphoproliferative disorders, plasmacytomas, histiocytomas, melanomas, adenomas, sarcomas, carcinomas of solid tissues, hypoxic tumors, squamous cell carcinomas, genitourinary cancers such as cervical and bladder cancer, hematopoietic cancers, head and neck cancers, and nervous system cancers.

The disclosure further includes the use of the modified cells described herein in the manufacture of a medicament or pharmaceutical composition to modulate an immune response, to treat an infection or to treat cancer as described hereinabove.

The modified cells can also be used in experimental models, for example, to further study and elucidate the function of the cells.

One or more of the modified cells described herein can be administered to a subject in a single, unified form, such as an intravenous injection, or in multiple forms, for example, as multiple intravenous infusions or injections, or subcutaneous injections. In some cases, the modified cells can expand within a subject's body, in vivo, after administration to a subject. The modified cells can be frozen to provide cells for multiple treatments with the same cell preparation. The modified cells of the disclosure, and pharmaceutical compositions comprising the same, can be packaged as a kit. A kit can include instructions (e.g., written instructions) on the use of the modified cells and compositions comprising the same.

Methods for administration of modified cells for adoptive cell therapy are known and can be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8 (10): 577-85). See, e.g., Themeli et al. (2013) Nat Biotechnol.31 (10): 928-933; Tsukahara et al. (2013) Biochem Biophys Res Commun 438 (1): 84-9; Davila et al. (2013) PLOS ONE 8 (4): e61338. The cell therapy, e.g., adoptive T cell therapy may be carried out by autologous transfer, in which the cells are isolated and/or otherwise prepared from the subject who is to receive the cell therapy, or from a sample derived from such a subject. Thus, in some aspects, the cells are derived from a subject, e.g., subject, in need of a treatment and the cells, following isolation and processing are administered to the same subject.

The cell therapy (e.g., adoptive T cell therapy), may be carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. The first and second subjects may be genetically identical. The first and second subjects may be genetically similar. The second subject may express the same HLA class or supertype as the first subject.

The subject may have been treated with a therapeutic agent targeting the disease or condition, e.g., the tumor, prior to administration of the cells or composition containing the cells. In some aspects, the subject is refractory or non-responsive to the other therapeutic agent. The subject may have persistent or relapsed disease, e.g., following treatment with another therapeutic intervention, including chemotherapy, radiation, and/or hematopoietic stem cell transplantation (HSCT), e.g., allogenic HSCT. The administration may effectively treat the subject despite the subject having become resistant to another therapy.

The subject may be responsive to the other therapeutic agent, and treatment with the therapeutic agent reduces disease burden. The subject may be initially responsive to the therapeutic agent, but exhibits a relapse of the disease or condition over time. The subject may have not relapsed. The subject may be determined to be at risk for relapse, such as at a high risk of relapse, and thus the cells are administered prophylactically, e.g., to reduce the likelihood of or prevent relapse. The subject may have not received prior treatment with another therapeutic agent.

The subject may have persistent or relapsed disease, e.g., following treatment with another therapeutic intervention, including chemotherapy, radiation, and/or hematopoietic stem cell transplantation (HSCT), e.g., allogenic HSCT. The administration may effectively treat the subject despite the subject having become resistant to another therapy.

The modified cells described herein can be administered to an animal, preferably a mammal, even more preferably a human, to treat a cancer. In addition, the modified cells can be used for the treatment of any condition related to a cancer, especially a cell-mediated immune response against a tumor cell(s), where it is desirable to treat or alleviate the disease. The types of cancers to be treated with the modified cells or pharmaceutical compositions include, carcinoma, blastoma, and sarcoma, and certain leukemia or lymphoid malignancies, benign and malignant tumors, and malignancies e.g., sarcomas, carcinomas, and melanomas. Other exemplary cancers include but are not limited breast cancer, prostate cancer, ovarian cancer, cervical cancer, skin cancer, pancreatic cancer, colorectal cancer, renal cancer, liver cancer, brain cancer, lymphoma, leukemia, lung cancer, thyroid cancer, and the like. The cancers can be non-solid tumors (such as hematological tumors) or solid tumors. Adult tumors/cancers and pediatric tumors/cancers are also included. The cancer may be a solid tumor or a hematological tumor. The cancer may be a carcinoma. The cancer may be a sarcoma. The cancer may be a leukemia. The cancer may be a solid tumor.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

Carcinomas that can be amenable to therapy by a method disclosed herein include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma.

Sarcomas that can be amenable to therapy by a method disclosed herein include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

The modified cells (e.g., immune cells, T cells, or NK cells) described herein can be included in a composition for immunotherapy. The composition can include a pharmaceutical composition and further include a pharmaceutically acceptable carrier. A therapeutically effective amount of the pharmaceutical composition comprising the modified cells can be administered.

The modified cells can be immediately used in the above therapeutic, experimental or commercial applications or the cells can be cryopreserved for use at a later date. The pharmaceutical compositions can be included in a container, pack, or dispenser together with instructions for administration.

The modified cells disclosed herein can be formulated in unit dosage forms suitable for single administration of precise dosages. In some cases, the unit dosage forms comprise additional lymphocytes. Cells can be expressed to the one or more of the plurality of output containers, e.g., vials, in an amount for dosage administration, such as for a single unit dosage administration or multiple dosage administration. For example, the vials may each contain the number of cells for administration in a given dose or fraction thereof. Thus, each vial may contain a single unit dose for administration or may contain a fraction of a desired dose such that more than one of the plurality of vials, such as two of the vials, or 3 of the vials, together constitute a dose for administration. Thus, the containers, e.g. bags or vials, generally contain the cells to be administered, e.g., one or more-unit doses thereof. The unit dose may be an amount or number of the cells to be administered to the subject or twice the number (or more) of the cells to be administered. It may be the lowest dose or lowest possible dose of the cells that would be administered to the subject.

### EXAMPLES

The disclosure is further described in the following examples, which do not limit the scope of the disclosure described in the claims.

### Example 1: Preparation of CAR-NK Cells Expressing Anti-BCMA CAR with Various Modified IL-15 Constructs

1.1 Preparation of Retrovirus Expression Vectors Containing Anti-BCMA CAR with Modified IL-15 Constructs

To construct the BCMA CAR (SEQ ID NO: 1), an anti-BCMA binder sequence was connected to CD8α hinge and transmembrane region followed by 4-1BB and CD3ζ intracellular sequence. The amino acid sequence of the BCMA CAR described above is shown in SEQ ID NO: 1.

The structures of the exemplary modified IL-15 constructs are shown in Tables 1-2. The amino acid sequences of the modified IL-15 constructs armored BCMA CAR are shown as SEQ ID NOs: 17-26.

To co-express modified IL-15 constructs, the polynucleotide encoding BCMA CAR was chemically synthesized and linked to the polynucleotide encoding modified IL-15 constructs by a coding sequence for a P2A linker (SEQ ID NO: 2).

The polynucleotide construct encoding the naked BCMA CAR is named CAR30. As shown in Tables 1-2, the polynucleotide constructs encoding both the BCMA CAR and modified IL-15 constructs are named CAR4, CAR5, CAR6, CAR11, CAR19, CAR20, CAR21, CAR22, and CAR32.

CAR31 refers to delta CAR (ACAR), which means that the modified cells only express introduced IL-15 (SEQ ID NO: 25).

Anti BCMA CAR with modified IL-15 constructs were synthesized in retrovirus vector SinCMV (Biovec pharma). Retrovirus were prepared by transfecting these constructs in HEK cell line (293 Vec-Galv™ containing stable transfection for gag-pol and env genes, Biovec pharma). Retroviruses generated from this cell line were then tittered using SupT1 cell line (ATCC, CRL1942). All constructs contain BCMA CAR connected to various designs of secreted wild type IL-15 module by P2A sequence. The amino acid sequences of the modified IL-15 constructs are listed in Table 1-2 below.

TABLE 1
The amino acid sequences of the modified IL-15 constructs

| | | | | IL-15 | | | |
|---|---|---|---|---|---|---|---|
| | | Signal | | Receptor | | ER | IL-15 |
| No. | IL-15 Design | peptide | IL-15 | Alpha (IL-15Rα) | Linker | retention | construct |
| 4 | CALRSP | SEQ ID | SEQ ID | — | SEQ ID NO: | SEQ ID | SEQ ID |
| | Linker1 | NO: 3 | NO: 6 | | 7 | NO: 13 | NO: 17 |
| | IL-15 | | | | SEQ ID NO: | | |
| | Linker2 | | | | 8 | | |
| | ER retention | | | | | | |
| 5 | IL-15SP | SEQ ID | SEQ ID | SEQ ID NO: | SEQ ID NO: | SEQ ID | SEQ ID |
| | IL-15 | NO: 4 | NO: 6 | 11 | 9 | NO: 12 | NO: 18 |
| | Linker | | | | | | |
| | IL-15Rα | | | | | | |
| | ER retention | | | | | | |
| 6 | CALRSP | SEQ ID | SEQ ID | SEQ ID NO: | SEQ ID NO: | SEQ ID | SEQ ID |
| | Linker1 | NO: 3 | NO: 6 | 11 | 7 | NO: 12 | NO: 19 |
| | IL-15 | | | | SEQ ID NO: | | |
| | Linker2 | | | | 9 | | |
| | IL-15Rα | | | | | | |
| | ER retention | | | | | | |
| 11 | CALRSP | SEQ ID | SEQ ID | SEQ ID NO: | SEQ ID NO: | SEQ ID | SEQ ID |
| | Linker1 | NO: 3 | NO: 6 | 10 | 7 | NO: 12 | NO: 20 |
| | IL-15 | | | | SEQ ID NO: | | |
| | Linker2 | | | | 9 | | |
| | IL-15Rα | | | | | | |
| | ER retention | | | | | | |

TABLE 2
The amino acid sequences of the modified IL-15 constructs

| | | | | | IL-15 | | |
|---|---|---|---|---|---|---|---|
| | | Signal | | | Receptor | | IL-15 |
| No. | Design | peptide | Myristoylation | IL-15 | Alpha (IL-15Rα) | Linker | construct |
| 19 | IL-15SP | SEQ ID | SEQ ID | SEQ ID | SEQ ID NO: | SEQ ID NO: | SEQ ID |
| | myristoy lation | NO: 4 | NO: 14 | NO: 6 | 11 | 9 | NO: 21 |
| | IL-15 | | | | | | |
| | Linker | | | | | | |
| | IL-15Rα | | | | | | |
| 20 | myristoylation | — | SEQ ID | SEQ ID | SEQ ID NO: | SEQ ID NO: | SEQ ID |
| | IL-15 | | NO: 14 | NO: 6 | 11 | 9 | NO: 22 |
| | Linker | | | | | | |
| | IL-15Rα | | | | | | |
| 21 | IL-15SP | SEQ ID | SEQ ID | SEQ ID | SEQ ID NO: | SEQ ID NO: | SEQ ID |
| | myristoylation | NO: 4 | NO: 15 | NO: 6 | 11 | 9 | NO: 23 |
| | IL-15 | | | | | | |
| | Linker | | | | | | |
| | IL-15Rα | | | | | | |
| 22 | IL-15SP | SEQ ID | SEQ ID | SEQ ID | SEQ ID NO: | SEQ ID NO: | SEQ ID |
| | myristoylation | NO: 4 | NO: 16 | NO: 6 | 11 | 9 | NO: 24 |
| | IL-15 | | | | | | |
| | Linker | | | | | | |
| | IL-15Rα | | | | | | |
| 31 | IL-15 | | | SEQ ID | | | SEQ ID |
| | | | | NO: 6 | | | NO: 25 |
| 32 | IL-15SP | SEQ ID | | SEQ ID | | | SEQ ID |
| | IL-15propeptide | NO: 4 | | NO: 5 | | | NO: 26 |
| | IL-15 | | | SEQ ID | | | |
| | | | | NO: 6 | | | |

### 1.2 NK Expansion and Virus Transduction of NK Cells

CD3 depleted PBMC cells derived from a healthy subject were cultured in a medium containing cytokine cocktail including IL2. The cells were transduced with retrovirus (generated from various constructs containing BCMA CAR and different modified IL-15 designs as shown in Tables 1-2) using spinocculation (MOI 1, 2000 g, 32° C., 1 h). Cells were harvested on Day 18. The cells were counted and stained with antibodies [CD56 (clone B159, BD Bioscience), CD3 (HIT3a, BD Bioscience), CD16 (clone3G8, BD Bioscience)] to calculate the NK expansion fold.

### Example 2: In Vitro Evaluation of IL-15 Construct Armored CAR-NK Cells

Various modified IL-15 construct armored CAR-NK cells were tested in three batches for various assays. Various in-vitro assays such as IL-15 ELISA, CAR detection assay, proliferation assay, cytotoxicity assay, and pSTAT5 assay were performed to evaluate various IL-15 designs in anti-BCMA CAR NK cells.

IL-15 ELISA: Secreted IL-15 from (1) medium and (2) NK cells lysate (from 4×10⁶ cells) as well as the ratio between secreted IL-15 and intracellular IL-15 were analyzed using ELISA kits (Ab218266, Abchem). Briefly, 50 μl of cocktail antibodies were coated in each well. Media control, lysate samples, and standards were diluted and 50 μl of the sample was added to each well. The plate was incubated for 1 h at room temperature (RT). The plate was washed three times with 350 μl wash buffer. Further, 100 μl of TMB (3,3′,5,5′-tetramethylbenzidine) substrate solution was added to each well. Plates were incubated for 10 min at RT and reaction was stopped with 100 μl of stop solution. Plates OD (optical density) at 450 nm was read using a spectrophotometer (Molecular devices).

CAR staining: The percentage of CAR positive cells in each analyzed construct was determined using immunophenotyping with BCMA-FITC protein (BCA-HF254, Acrobiosystems Inc). Briefly, 2×10⁵ harvested NK cells were washed and re-suspended in 100 μl staining buffer (PBS+2% (w/v) BSA) containing FITC labeled BCMA protein (3 μg/ml) and were added to each well in a 96 well plate. The plate was incubated at 4° C. for 1 h. Further, the plate was washed with a wash buffer, and 150 μl of diluted 7AAD (7-Aminoactinomycin D) solution (5 μl 7AAD per sample, BD Bioscience) was added. Samples were analyzed on flowcytometer (Aurora, Cytek Biosciences) and CAR-FITC positive cells were determined.

pSTAT5 assay: phosphorylated STAT5 (pSTAT5) levels were evaluated using a pSTAT5 specific antibody. Briefly, 0.5×10⁶ NK cells were added to each well and stained with CD56 (clone5.1H11, Biolegend), BCMA-FITC (BCA-HF254, Acrobiosystems Inc) and NIR live dead dye (L10119, Invitrogen) for 30 min. Further, cells were fixed with 1.5% (w/v) para-formaldehyde (PFA) at 37° C. in incubator for 10 min. PFA fixed cells were centrifuged, and the pellet was re-suspended and fixed with 1 mL ice-cold methanol for 1 h at 4° C. Fixed cells were washed and re-suspended in staining buffer containing a pSTAT5 antibody (pY694 Cat no 612599, BD Bioscience) for 1 h at RT. Cells were analyzed for pSTAT5 signal using a flowcytometer (Aurora, Cytek Biosciences).

Proliferation assay: Cellular proliferation was analyzed either by counting the absolute number of cells using trypan blue (Vi cell Blue, Beckman Coulter) or by the dye dilution method. For the dye dilution method, NK cells were washed twice in PBS and incubated with cell trace violet (4 μM, Invitrogen) for 20 min at 37° C. Serum was added to stop the reaction, cells were washed and re-suspended in culture media. Further, cells were incubated for a certain period in cell culture incubator. Cells were harvested at the end of the certain period and analyzed for dilution of cell trace violet dye using flowcytometer (Aurora, Cytek Biosciences).

Cytotoxicity assay: Cytotoxic activity of NK cells was evaluated using a short term 4 h cytotoxicity assay. Briefly, tumor cells were labeled with cell trace violet dye (4 μM, Invitrogen) as described above in the proliferation assay. NK cells and labeled tumor cells were washed, counted, and adjusted to 1×10⁶/ml. Based on the E/T ratio 1:1, 0.5:1, 0.25:1, 0.12:1, NK cells were plated first using a serial dilution with a dilution factor of 2. Further, appropriate numbers of tumor cells were plated in respective wells to set-up the co-culture. Plates were centrifuged briefly at 100 g for 1 min and incubated for 4 h in a cell culture incubator. Plates were washed and stained with anti-CD3 (clone HIT3a, BD Bioscience), CD56 (clone B159, BD Bioscience), CD16 (clone3G8, BD Bioscience) and BCMA-FITC protein (BCA-HF254, Acrobiosystems Inc) for 30 min at 4° C. Plates were washed and re-suspended in 150 μl 7AAD solution (5 μl 7AAD per sample). Samples were acquired and analyzed using a flowcytometer (Aurora, Cytek Biosciences).

Results from various assays have been summarized for Batch 1 in Table 4 below, Batch2 in Table 5 below, and Batch 3 in Table 6 below.

TABLE 4
Various assay results for constructs tested in Batch 1

| | NK | NK | | | | | | Proliferation | |
|---|---|---|---|---|---|---|---|---|---|
| | medium | lysate | | | | pSTAT5+ | Proliferation | by Dye | |
| | (pg/4M | (pg/4M | | | % CAR+ | MFI | by Cell | dilution | % Cytotoxicity |
| ID | cells) | cells) | Ratio | % CAR+ | pSTAT5+ | (CAR+) | number (10⁶) | (MFI) | (E/T = 0.5) |
| CAR11 | 1415.1 | 186 | 7.6 | 81.3 | 86.6 | 7376 | 2.5 | 70% | 39.5 |
| CAR19 | 199.8 | 38.7 | 5.2 | 44.3 | 59 | 5661 | 0.799 | 56% | 36.3 |
| CAR20 | 99.7 | 38.8 | 2.6 | 38.7 | 56 | 5427 | 1.03 | 66% | 37.9 |
| CAR32 | 1202.8 | 137.6 | 8.7 | 76 | 76.7 | 7003 | 4.01 | 85% | 30.5 |

TABLE 5
Various assay results for constructs tested in Batch 2

| | NK | NK | | | | | | | |
|---|---|---|---|---|---|---|---|---|---|
| | medium | lysate | | | | pSTAT5+ | Proliferation | Proliferation by | % |
| | (pg/4M | (pg/4M | | % | % CAR+ | MFI | by Cell | Dye dilution | Cytotoxicity |
| ID | cells) | cells) | Ratio | CAR+ | pSTAT5+ | (CAR+) | number (10⁶) | (MFI) | (E/T = 0.5) |
| CAR5 | 398.5 | 108.5 | 3.7 | 70.8 | 56.4 | 5568 | 3.34 | 88% | 51.9 |
| CAR21 | 46 | 20.5 | 2.2 | 25.5 | 19.2 | 3237 | 0.648 | 68% | 48.2 |
| CAR31 | 2332.5 | 357.5 | 6.5 | 0.45 | 69.9 | 6181 | N/A | N/A | N/A |

TABLE 6
Various assay results for constructs tested in Batch 3

| | NK | NK | | | | | Proliferation | Proliferation | |
|---|---|---|---|---|---|---|---|---|---|
| | medium | lysate | | | | pSTAT5+ | by Cell | by Dye | % |
| | (pg/4M | (pg/4M | | | % CAR+ | MFI | number | dilution | Cytotoxicity |
| ID | cells) | cells) | Ratio | % CAR+ | pSTAT5+ | (CAR+) | (10⁶) | (MFI) | (E/T = 0.5) |
| CAR4 | 366 | 2223.5 | 0.16 | 40 | 47 | 1178 | 1.9 | 96% | 77 |
| CAR6 | 53 | 109 | 0.49 | 28 | 52 | 1266 | 1.44 | 94% | 79.10 |
| CAR22 | 58.5 | 121.5 | 10.48 | 28 | 40 | 931 | 1.24 | 92% | 80.30 |

### Example 3: In Vitro Evaluation of CAR5, CAR6, CAR19, CAR20 and CAR22

Various in-vitro assays such as CAR detection assay, proliferation assay, cytotoxicity assay, and pSTAT5 assay were performed as described above in Example 2 to evaluate various modified IL-15 designs in anti-BCMA CAR NK cells.

### 3.1 Immunostaining Assay

Modified IL-15 constructs armored BCMA CAR-NK cells were tested for cell surface IL-15 and IL-15Rα expression with antibodies against IL-15 cytokine (clone34559, Invitrogen) and IL-15 receptor alpha (clone 2639B, Biotechne) on Day 18 using a flow cytometer. Briefly, 0.1×10⁶ cells were taken from each well of a 96 well plate. Cells were washed, blocked with a Fc antibody for 10 min and stained with IL-15 and IL-15Rα antibodies for 45 min on ice at 4° C. Samples were washed and labeled with diluted live dead dye 7AAD (5 μl 7AAD per sample, BD Bioscience). Samples were acquired and analyzed using a flowcytometer (Aurora, Cytek Biosciences). CAR32 transduced with BCMA and wild type IL15 construct was used as control.

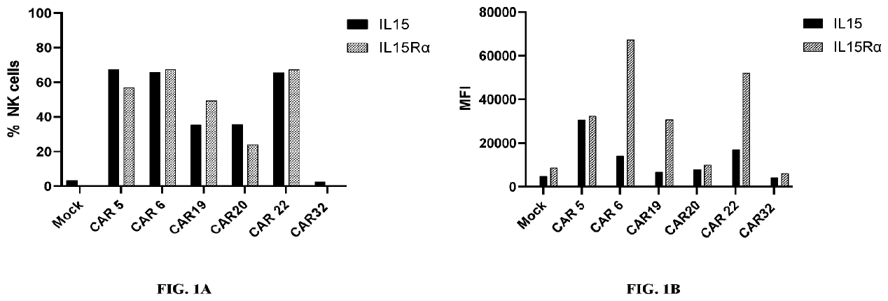

As shown in FIG. 1, CAR6 and CAR22 armored BCMA CAR-NK cells showed higher positive percentages levels of cell surface IL-15 and IL-15Rα compared to CAR32 and other modified IL-15 constructs armored BCMA CAR-NK cells.

Modified IL-15 construct armored BCMA CAR-NK cells were tested for the CAR positive percentages using a BCMA-FITC protein (BCA-HF254, Acrobiosystems Inc). CAR32 transduced with BCMA and wild type IL15 construct was used as control.

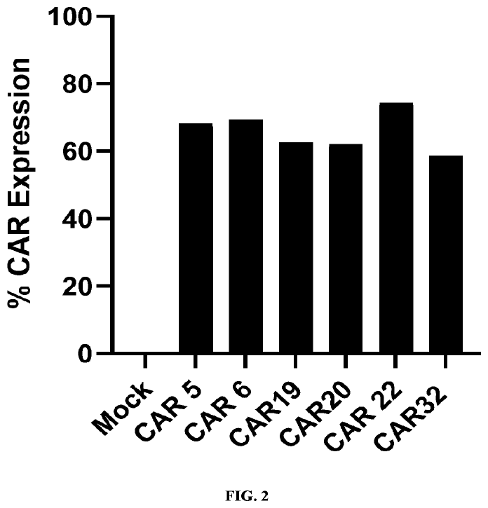

As shown in FIG. 2, all modified IL-15 constructs armored BCMA CAR-NK cells showed over 55% CAR positive percentage.

### 3.2 Proliferation Assay

To analyze the effects of different modified IL-15 constructs on cell proliferation, the modified IL-15 constructs armored BCMA CAR-NK cells were tested for the total number of cells. Cell cultures were seeded with equal number of cells 1×10⁶ cells per well on Day 0 in cytokine free medium and were counted on Day 9. Cell numbers in million and viability were plotted. CAR32 transduced with BCMA and wild type IL15 construct was used as control.

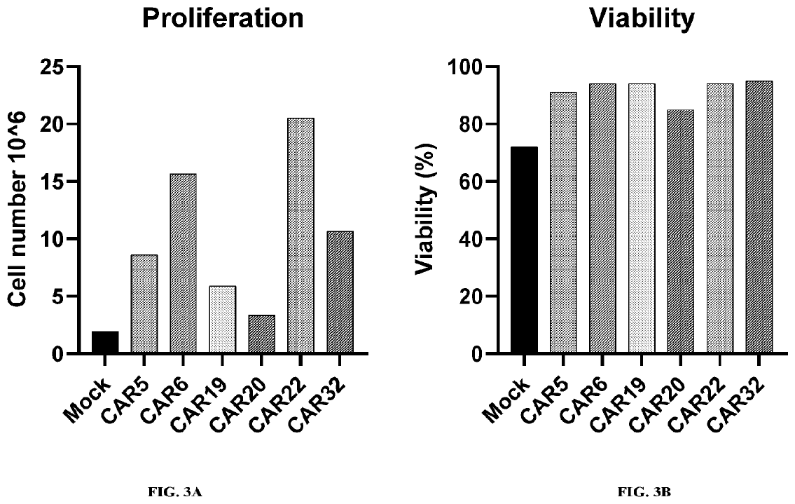

As shown in FIGS. 3A-3B, comparing to other IL-15 construct armored BCMA CAR-NK cells, CAR6 and CAR22 armored BCMA CAR-NK cells showed higher proliferation.

Further, the modified IL-15 constructs armored BCMA CAR-NK cells were tested for cellular proliferation using the dye dilution method. Cell trace violet (4 μM, Invitrogen) labeled cells in equal number 1×10⁶ per well were seeded on Day 0 in cytokine free medium and were evaluated for dye dilution on Day 9. Decrease in MFI and fold proliferation were plotted for each construct design. CAR32 transduced with BCMA and wild type IL15 construct was used as control.

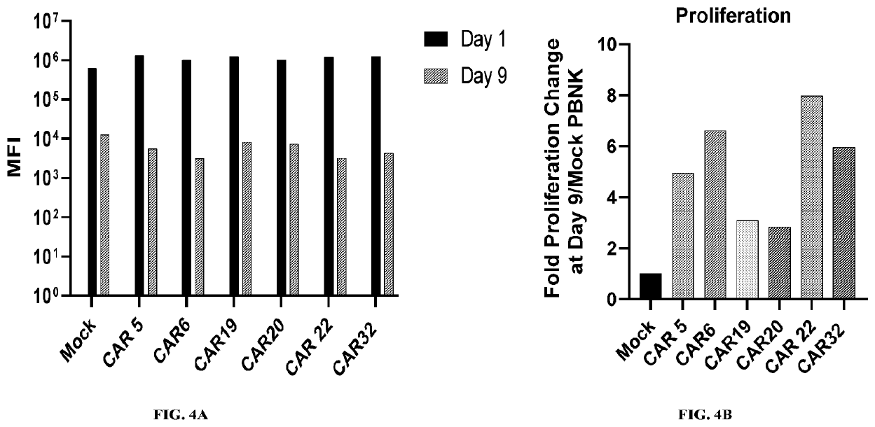

As shown in FIGS. 4A-4B, comparing to CAR32 and other IL-15 construct armored BCMA CAR-NK cells, CAR6 and CAR22 armored BCMA CAR-NK cells showed higher proliferation.

3.3 Percentage Activity by pSTAT5 Expression

To analyze the effects of different modified IL-15 constructs on STAT5 signaling, the modified IL-15 constructs armored BCMA CAR-NK cells were tested for phosphorylated STAT5 (the percentages of pSTAT5 positive cells and MFI) using the method described above. CAR32 transduced with BCMA and wild type IL15 construct was used as control.

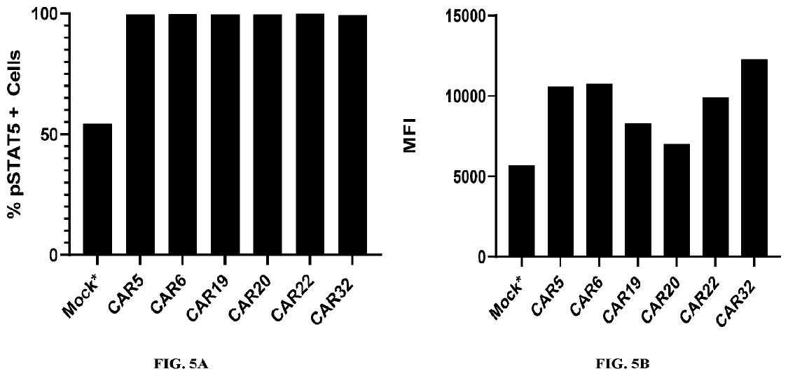

As shown in FIGS. 5A-5B, all modified IL-15 constructs armored BCMA CAR-NK cells showed lower STAT5 activation activities. Comparing to CAR32, CAR6 and CAR22 armored BCMA CAR-NK cells, which proliferated well, also showed lower STAT5 activation activities.

### 3.4 Cytotoxic Activity by Serial Cytotoxicity Assay

Modified IL-15 constructs armored BCMA CAR-NK cells were tested for cell killing activities by a serial cytotoxicity assay against H929 (NCI-H929) cells (E:T=0.25). Briefly, a co-culture of NK cells and cell trace violet (4 μM, Invitrogen) labeled tumor (NCI-H929) cells was established and analyzed using method described above regarding the cytotoxicity assay. Freshly cell trace violet labeled H929 cells were added every 24 h to evaluate cytotoxicity in a repeat challenge assay, where the modified NK cells were exposed to multiple rounds of tumor cell challenge. Each new addition of tumor cells exposure was defined as one round and cells were analyzed after each round of challenge. Live tumor cell measurements were analyzed to calculate percent cytotoxicity and percent CAR after each round of tumor exposure. CAR32 transduced with BCMA and wild type IL15 construct was used as control.

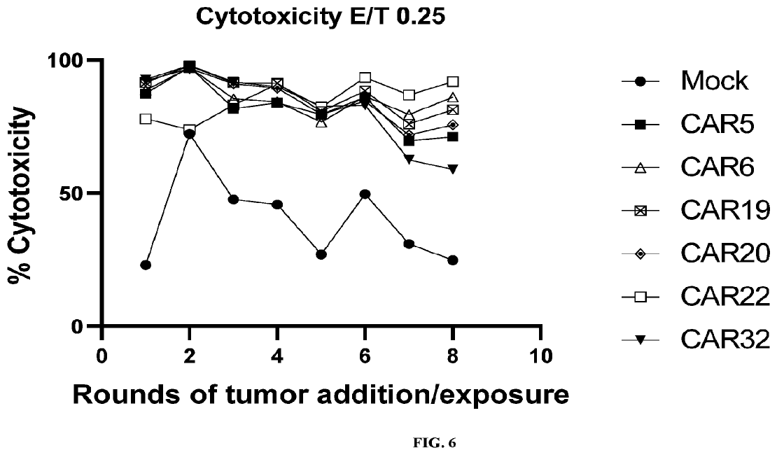

As shown in FIG. 6, comparing to CAR32 and other IL-15 construct armored BCMA CAR-NK cells, CAR6 and CAR22 armored BCMA CAR-NK cells showed the highest cell killing activities after round 8 of tumor cells exposure.

### 3.5 Safety Assay

To analyze the safety of modified IL-15 constructs armored BCMA CAR-NK cells, the modified IL-15 constructs armored BCMA CAR-NK cells were tested for proliferation in absence of tumor antigen in cytokine free medium. Cell cultures with equal number of 1×10⁶ cells per well were seeded on Day 0 in cytokine free medium and were counted until Day 49, after the cells were stained using trypan blue (Vi cell Blue, Beckman Coulter). Cell numbers in million and viability were plotted. CAR32 transduced with BCMA and wild type IL15 construct was used as control.

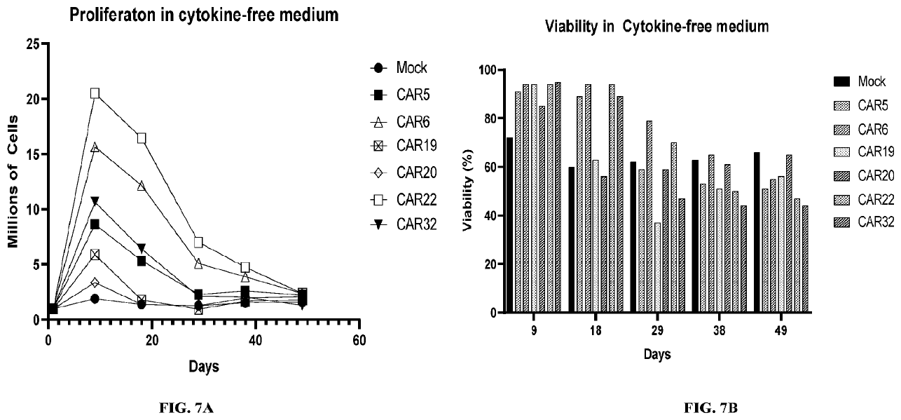

As shown in FIGS. 7A-7B, all IL-15 construct armored BCMA CAR-NK cells expanded until Day 9 and then cell numbers and viability gradually decreased until Day 49.

### Example 4: In Vivo Evaluation of CAR5, CAR6, CAR19, CAR20 and CAR22

The in vivo tumor suppression effects of CAR NK cells with selected modified IL-15 designs were evaluated in a tumor xenograft NCG mouse model. The experiment was designed with 9 groups with 4 NCG mice per group. Mice in a blank group only received PBS. To create the tumor xenograft, NCG mice of all other groups were injected intravenously with 2 million Luciferase labeled NCI-H929 tumor cells (#ATCC CRL-9068).

Twelve days post-tumor engraftment, mice were injected intravenously (i.v.) with 2 million IL-15 construct armored CAR-NK cells or unNK (untransduced NK) cells. Tumor progression was monitored weekly using in vivo bioluminescence imaging (BLI). Blood was collected from the mice weekly, and the percentage of human NK cells and the number of human CAR-NK cells were determined by flow cytometric analysis. Body weight was also measured at each time point. Mice were then observed for survival for up to 50 days post-treatment.

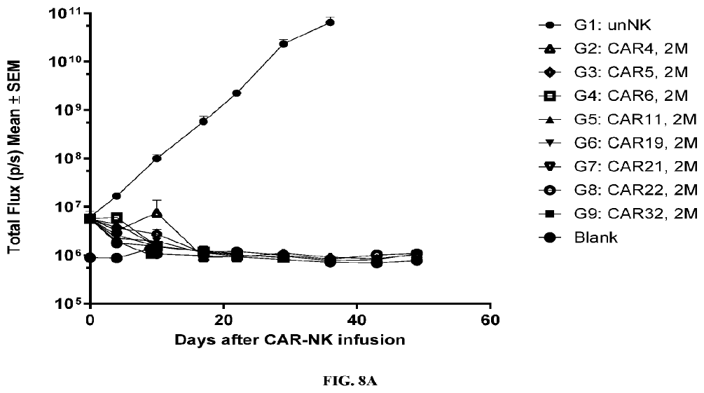

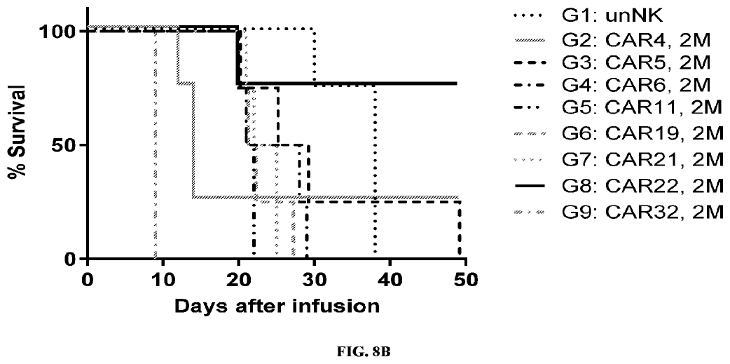

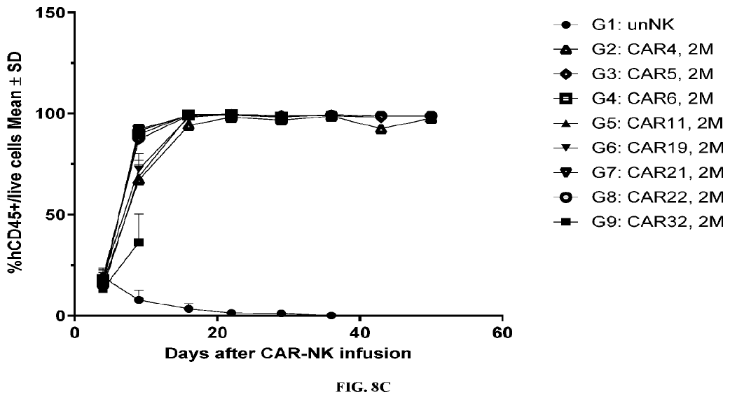

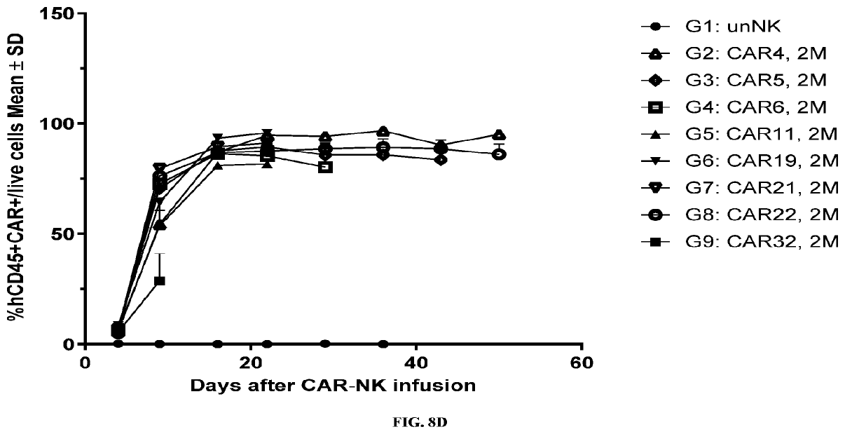

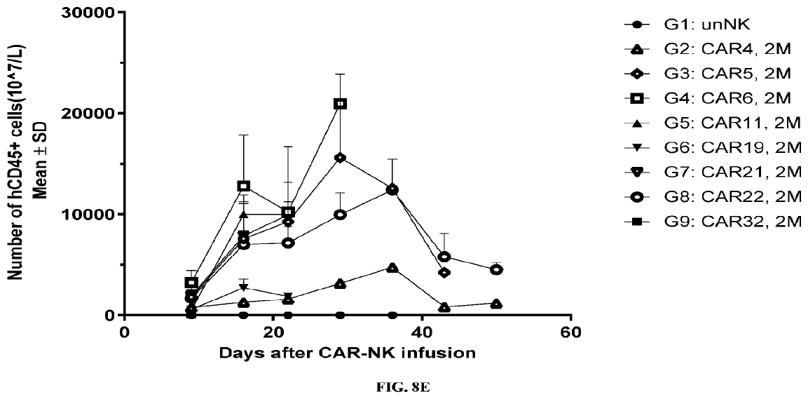

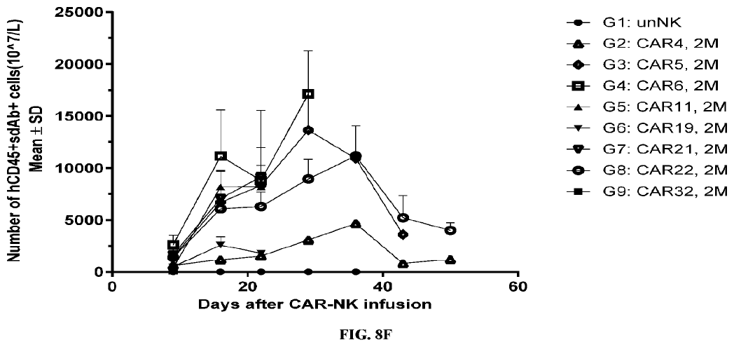

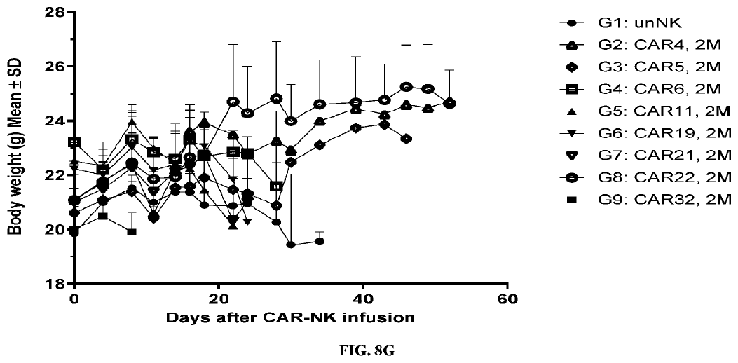

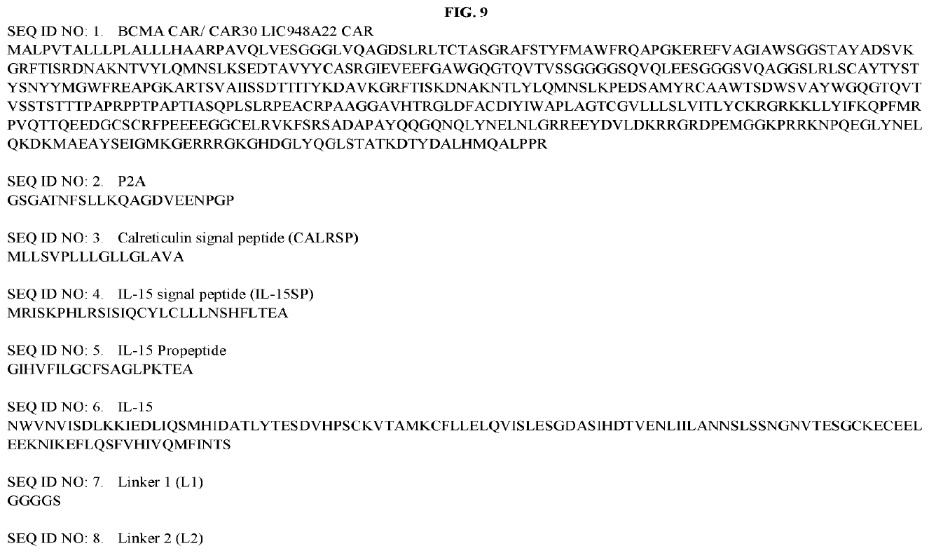

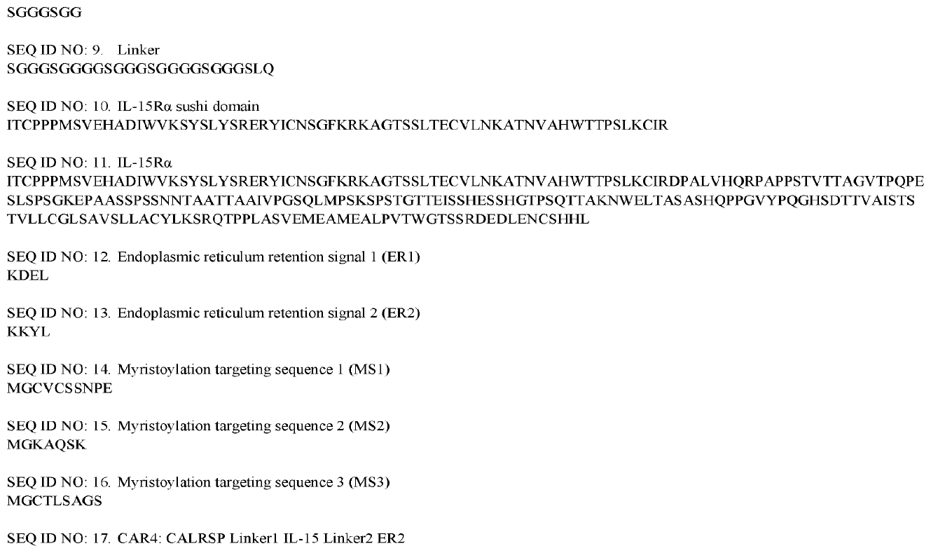

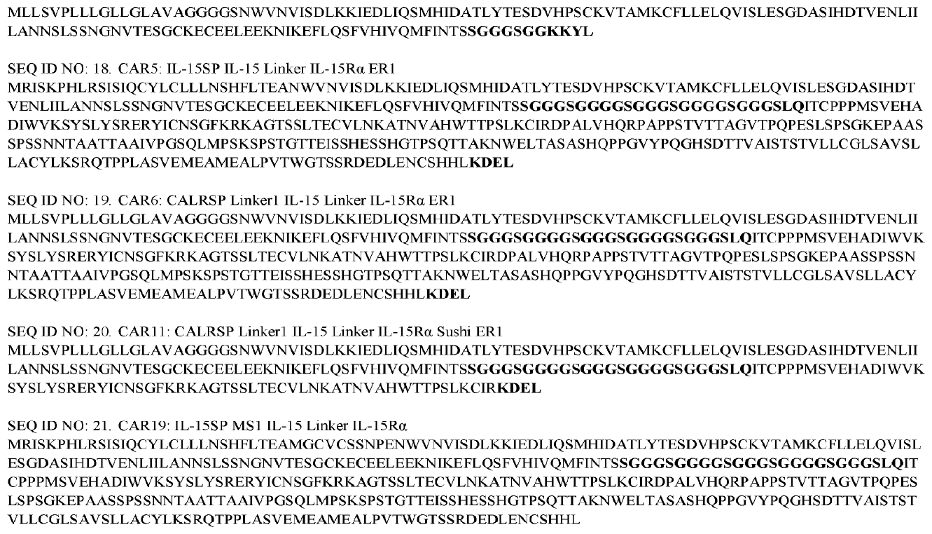

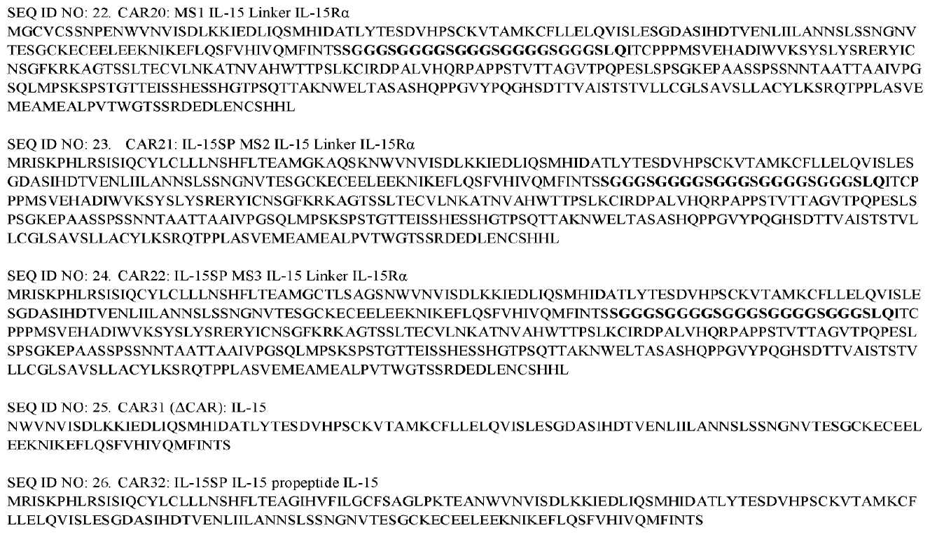

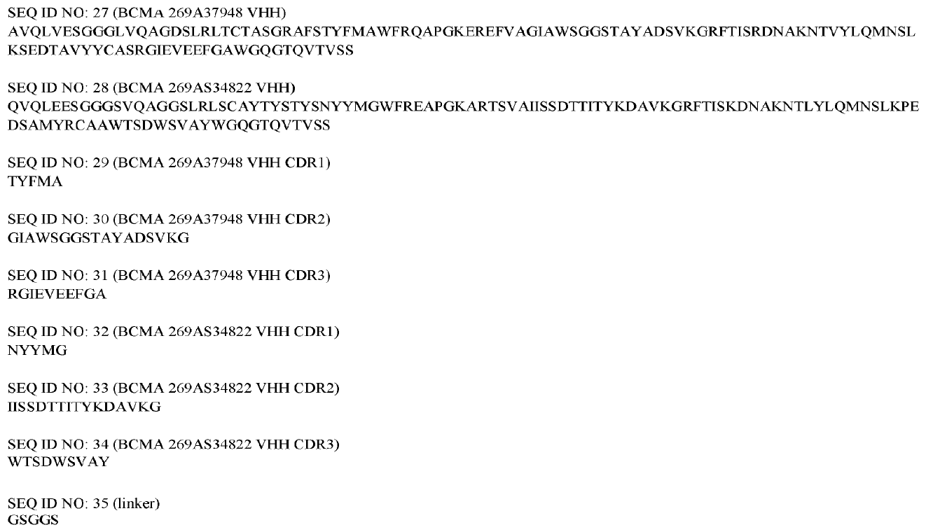

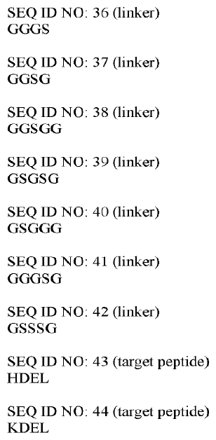

Our data demonstrate that mice treated with anti-BCMA CAR containing wild type IL15 construct (CAR32) showed toxicity with mice dying at day 9. Construct 22 had the highest potential for tumor elimination, with reduced tumor levels comparable to that of blank mice (FIG. 8A). One mouse in each of the Construct 4 and Construct 5 groups also had a reduced tumor burden (FIG. 8A). Construct 22 derived CAR-NK cells showed the highest activity in eliminating tumors, and highest survival with mice surviving up to 50 days (FIG. 8B). One mouse with construct 5 survived until day 43, while one mouse with construct 4 survived until day 50. Analysis of total CD45 positive NK cells as well as CAR positive NK cells showed that constructs 22 had the highest expansion of percentage (FIGS. 8C-8D) and absolute numbers (FIGS. 8E-8F) in mice. No significant change in body weight as well as fur of mice were observed in this experiment (FIG. 8G), suggesting that GVHD did not occur in this experiment. While one mouse with construct 4 survived until end of experiment, three mice with construct 22 survived until end of experiment. This suggests that construct 22 design of the IL15 module supported effective killing of tumor cells without compromising safety, construct 22 is effective and safe.

### OTHER EMBODIMENTS

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

What is claimed is:

1. A fusion protein comprising
(1) a signal peptide sequence;
(2) an interleukin-15 (IL-15) sequence; and
(3) an endoplasmic reticulum (ER) retention sequence.

2. The fusion protein of claim 1, wherein the signal peptide sequence is calreticulin signal peptide sequence.

3. The fusion protein of claim 2, wherein the calreticulin signal peptide sequence comprising an amino acid sequence set forth in SEQ ID NO: 3, or an amino acid sequence having at least 90%, 95%, or 99% identity to the amino acid sequence set forth in SEQ ID NO: 3.

4. The fusion protein of any one of claims 1-3, wherein the ER retention sequence comprises an amino acid sequence set forth in SEQ ID NO: 12 or 13, or an amino acid sequence having at least 90%, 95%, or 99% identity to the amino acid sequence set forth in SEQ ID NO: 12 or 13.

5. A fusion protein comprising
(1) a cell membrane targeting sequence; and
(2) an IL-15 sequence.

6. The fusion protein of claim 5, wherein the cell membrane targeting sequence is selected from the group consisting of a myristoylation sequence, a palmitoylation sequence, and a prenylation sequence.

7. The fusion protein of claim 5 or 6, wherein the cell membrane targeting sequence is a myristoylation sequence.

8. The fusion protein of claim 7, wherein the myristoylation sequence comprises an amino acid sequence set forth in any one of SEQ ID NOs: 14-16, or an amino acid sequence having at least 90%, 95%, or 99% identity to the amino acid sequence set forth in any one of SEQ ID NOs: 14-16.

9. The fusion protein of any one of claims 1-8, wherein the IL-15 sequence comprises an amino acid sequence set forth in SEQ ID NO: 6, or an amino acid sequence having at least 90%, 95%, or 99% identity to the amino acid sequence set forth in SEQ ID NO: 6.

10. The fusion protein of any one of claims 1-9, wherein the fusion protein further comprises an IL-15Rα sequence.

11. The fusion protein of claim 10, wherein the IL-15Rα sequence comprises an amino acid sequence set forth in SEQ ID NO: 11, or an amino acid sequence having at least 90%, 95%, or 99% identity to the amino acid sequence set forth in SEQ ID NO: 11.

12. The fusion protein of claim 10 or 11, wherein the IL-15Rα sequence comprises or consists of an IL-15Rα Sushi domain, wherein the IL-15Rα Sushi domain comprises or consists of an amino acid sequence set forth in SEQ ID NO: 10, or an amino acid sequence having at least 90%, 95%, or 99% identity to the amino acid sequence set forth in SEQ ID NO: 10.

13. The fusion protein of any one of claims 1-4 and 9-12, wherein the fusion protein comprises, from N-terminus to C-terminus, a signal peptide sequence, a first linker sequence, an IL-15 sequence, a second linker sequence, an IL-15Rα sequence, and an ER retention sequence.

14. The fusion protein of any one of claims 1-4 and 9-13, wherein the fusion protein comprises an amino acid sequence set forth in any one of SEQ ID NOs: 18-20, or an amino acid sequence having at least 90%, 95%, or 99% identity to the amino acid sequence set forth in any one of SEQ ID NOs: 18-20.

15. The fusion protein of claim 14, wherein the fusion protein comprises an amino acid sequence set forth in SEQ ID NO: 19, or an amino acid sequence having at least 90%, 95%, or 99% identity to the amino acid sequence set forth in SEQ ID NO: 19.

16. The fusion protein of any one of claims 5-12, wherein the fusion protein comprises, from N-terminus to C-terminus, an optional signal peptide sequence, a myristoylation sequence, an IL-15 sequence, a linker sequence, and an IL-15Rα sequence.

17. The fusion protein of any one of claims 5-12 and 16, wherein the fusion protein comprises an amino acid sequence set forth in any one of SEQ ID NOs: 21-24, or an amino acid sequence having at least 90%, 95%, or 99% identity to the amino acid sequence set forth in any one of SEQ ID NOs: 21-24.

18. The fusion protein of claim 17, wherein the fusion protein comprises an amino acid sequence set forth in SEQ ID NO: 24, or an amino acid sequence having at least 90%, 95%, or 99% identity to the amino acid sequence set forth in SEQ ID NO: 24.

19. A nucleic acid comprising one or more nucleic acid sequences encoding the fusion protein of any one of claims 1-18 or a portion thereof.

20. The nucleic acid of claim 19, wherein the nucleic acid further comprises a second nucleic acid sequence encoding an engineered receptor, wherein the engineered receptor comprises an extracellular antigen binding domain or ligand binding domain, and optionally an intracellular signaling domain.

21. The nucleic acid of claim 20, wherein the engineered receptor nucleic acid sequence and the nucleic acid sequences encoding the fusion protein are separated by a third nucleic acid sequence encoding a cleavable linker.

22. The nucleic acid of claim 21, wherein the cleavable linker comprises an amino acid sequence set forth in SEQ ID NO: 2, or an amino acid sequence having at least 90%, 95%, or 99% identity to the amino acid sequence set forth in any one of SEQ ID NO: 2.

23. The nucleic acid of any one of claims 20-22, wherein the engineered receptor is selected from the group consisting of an engineered T cell receptor (TCR), a chimeric antigen receptor (CAR), a T cell antigen coupler (TAC) or a portion thereof.

24. The nucleic acid of any one of claims 20-23, wherein the engineered receptor is a CAR.

25. The nucleic acid of claim 23 or 24, wherein the CAR comprises an extracellular antigen binding domain that specifically binds to an antigen, wherein the antigen is a tumor antigen selected from the group consisting of CD19, CD20, CD22, CD30, CD33, CD38, BCMA, CS1, CD138, CD123/IL3Rα, c-Met, gp100, MUC1, IGF-I receptor, EpCAM, EGFR/EGFRvIII, HER2, IGF1R, mesothelin, PSMA, WT1, ROR1, CEA, GD-2, NY-ESO-1, MAGE A3, DLL3, GPC3, guanylate cyclase 2C (GCC), Claudin18.2, Claudin6, Glycolipid F77, PD-L1, and/or PD-L2.

26. The nucleic acid of claim 25, wherein the tumor antigen is BCMA.

27. The nucleic acid of any one of claims 23-26, wherein the CAR comprises a first VHH antibody moiety that comprises a CDR1 comprising the amino acid sequence of SEQ ID NO: 29, a CDR2 comprising the amino acid sequence of SEQ ID NO: 30, and a CDR3 comprising the amino acid sequence of SEQ ID NO: 31, and a second VHH antibody moiety that comprises a CDR1 comprising the amino acid sequence of SEQ ID NO: 32, a CDR2 comprising the amino acid sequence of SEQ ID NO:33, and a CDR3 comprising the amino acid sequence of SEQ ID NO: 34.

28. The nucleic acid of any one of claims 23-27, wherein the CAR comprises a first VHH antibody moiety comprising the amino acid sequences of SEQ ID NO: 27 or an amino acid sequence having at least 90%, 95%, or 99% identity to the amino acid sequence of SEQ ID NO: 27, and a second VHH antibody moiety comprising the amino acid sequences of SEQ ID NO: 28 or an amino acid sequence having at least 90%, 95%, or 99% identity to the amino acid sequence of SEQ ID NO: 28.

29. The nucleic acid of any one of claims 23-28, wherein the CAR comprises an amino acid sequence set forth in SEQ ID NO: 1, or an amino acid sequence having at least 90%, 95%, or 99% identity to the amino acid sequence set forth in any one of SEQ ID NO: 1.

30. A vector comprising the nucleic acid of any one of claims 19-29.

31. A cell comprising the fusion protein of any one of claims 1-18, the nucleic acid of any one of claims 19-29, and/or the vector of claim 30.

32. The cell of claim 31, wherein the cell expresses an engineered receptor.

33. The cell of claim 32, wherein the engineered receptor is selected from the group consisting of an engineered T cell receptor (TCR), a chimeric antigen receptor (CAR), a T cell antigen coupler (TAC) or a portion thereof.

34. The cell of claim 32 or 33, wherein the engineered receptor specifically recognizes a tumor antigen.

35. The cell of any one of claims 31-34, wherein the cell an immune cell.

36. The cell of any one of claims 31-34, wherein the cell is selected from a group consisting of T cell, αβT cell, γδT cell, NK cell, peripheral blood mononuclear cell (PBMC), hematopoietic stem cell, pluripotent stem cell, an embryonic stem cell, and a combination thereof.

37. The cell of claim 36, wherein the cell is a NK cell.

38. A method for producing the cell of any one of claims 31-37, the method comprising introducing the vector of claim 30 into a cell.

39. A method of treating a subject having cancer, the method comprising administering to the subject in need thereof a therapeutically effective amount of the cell of any one of claims 31-37.

40. The method of claim 39, wherein the subject has breast cancer, lung cancer, pancreatic cancer, melanoma, oral cancer, mesothelioma, ovarian cancer, colorectal cancer, gastric cancer, cervical cancer, brain cancer, skin cancer, multiple myeloma, lymphoma, epithelial neoplasms, soft tissue sarcoma, esophageal cancers, or CNS tumors.

41. The fusion protein of any one of claims 1-18, wherein the fusion protein is trapped inside the cells to reduce the amount of secreted IL-15 and therefore reduce the toxicity of IL-15.
